(19) United States
(12) Patent Application Publication
(10) Pub. No.: US 2006/0204874 A1
(43) Pub. Date: Sep. 14, 2006

(54) Quinone compound, electrophotographic photoconductor, and electrophotographic apparatus

(75) Inventors: Kenichi Ohkura, Matsumoto (JP); Motohiro Takeshima, Minami-Alps (JP); Shinichi Omokawa, Matsumoto (JP); Yoshiki Hasegawa, Chigasaki City (JP); Tohru Kobayashi, Hiratsuka City (JP)

Correspondence Address: SUGHRUE MION, PLLC, 2100 PENNSYLVANIA AVENUE, N.W., SUITE 800, WASHINGTON, DC, 20037, US

(73) Assignees: FUJI ELECTRIC IMAGING DEVICE CO., LTD.; TAKASAGO INTERNATIONAL CORPORATION

(21) Appl. No.: 11/374,031

(22) Filed: Mar. 14, 2006

(30) Foreign Application Priority Data
Mar. 14, 2005 (JP) 071267/2005

(51) Int. Cl. G03G 5/06; C07C 245/04

(52) U.S. Cl. 430/70; 534/744

(57) ABSTRACT

A compound having a superior electron-transporting property useful for electrophotographic photoconductors and organic ELs, a positive charge type electrophotographic photoconductor for high sensitive copiers or printers and an electrophotographic apparatus using the same, by using the organic compound in a photosensitive layer as an electron-transporting material are provided.
The present invention relates a quinone compound having a structure represented by Formula (I): 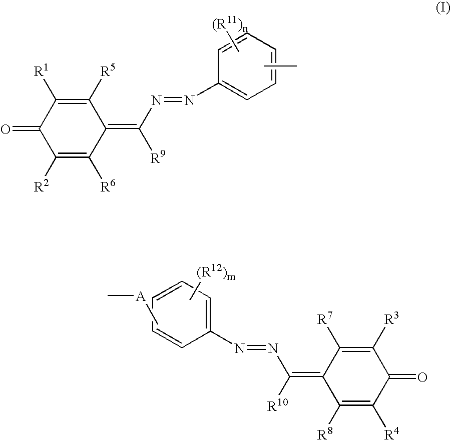
(wherein R¹, R², R³, R⁴, R⁵, R⁶, R⁷ and R⁸ each denote hydrogen or an alkyl; R⁹ and R¹⁰ each denote hydrogen, an alkyl, aryl, or heterocyclic group; R¹¹ and R¹² each denote a halogen, an alkyl, alkoxy, alkyl halide, nitro, aryl, or heterocyclic group; n and m each denote an integer of 0 to 4; A denotes oxygen or SO₂; and the substituent is a halogen, an alkyl, alkoxy, alkyl halide, nitro, aryl, or heterocyclic group), an electrophotographic photoconductor, and an electrophotographic apparatus using the same.

### BACKGROUND OF THE INVENTION

1. Field of the Invention

The present invention relates to novel quinone compounds, more specifically, relates to novel quinone compounds useful as charge-transporting materials for electrophotographic photoconductors (hereinafter also referred to as simply “photoconductors”). Furthermore, the present invention relates to electrophotographic photoconductors and electrophotographic apparatuses, more specifically, relates to electrophotographic photoconductors used in electrophotographic printers or copiers provided with photosensitive layers including organic materials on electrically conductive substrates and relates to electrophotographic apparatuses using the same.

2. Description of the Related Art

Conventionally, inorganic photoconductive materials such as selenium and selenium alloys or inorganic photoconductive materials such as zinc oxide and cadmium sulfide dispersed in resin binders have been used as photosensitive layers of electrophotographic photoconductors. In recent years, electrophotographic photoconductors using organic photoconductive materials have been studied, and some of them, which have been improved in sensitivity and durability, are in practical use.

The photoconductors must be capable of retaining surface charges in the dark, absorbing light to generate charges, and likewise absorbing light to transport charges. In the photoconductors, there are single-layer photoconductors in which one layer satisfies these functions and multilayer photoconductors in which the functions are separated into a layer mainly contributing for generation of charges and a layer contributing for charge retention in the dark and for charge-transportation when light is absorbed.

In image-forming by means of electrophotography using these photoconductors, the Carson process is utilized, for example. In this process, the image forming is conducted by charging the photoconductor by corona discharge in the dark, forming an electrostatic latent image such as characters and pictures of a copy on the surface of the charged photoconductor, developing the formed electrostatic latent image with toner, and photographically fixing the developed toner image on a carrier such as paper. The photoconductor after the toner transfer is reused by removing electricity, residual toner, and discharge by light.

Organic photoconductors in practical use have advantages, compared to inorganic photoconductors, in flexibility, film formability, and safety, and are being further studied to improve sensitivity and durability because of the variety of the materials.

Most organic photoconductors are multilayer photoconductors of which functions are separated into a charge-generating layer and a charge-transporting layer. Generally, the multilayer organic photoconductors are each provided by forming a charge-generating layer containing a charge-generating material such as pigments and dyes and a charge-transporting layer containing a charge-transporting material such as hydrazone and triphenylamine in this order on an electrically conductive substrate. The multilayer organic photoconductors are a hole-transporting type because the charge-transporting materials are an electron donor. Consequently, the organic photoconductors have sensitivity when their surfaces are negatively charged. However, in the negative charge type, corona discharge used in the charging is unstable compared with that in a positive charge type. Additionally, since ozone and nitrogen oxides are generated, they adhere to the photoconductor surfaces to easily cause physical and chemical degradation and also deteriorate the environment, which are problems. Based on these points, the positive charge type photoconductors, which have a high flexibility in use conditions, are more advantageous and widely applicable than the negative charge type photoconductors.

Therefore, in order to use as a positive charge type photoconductor, there has been proposed to use a charge-generating material and a charge-transporting material simultaneously dispersed in a resin binder as a single-layer photosensitive layer. Some of such single-layer photoconductors are in practical use. However, the single-layer photoconductors have insufficient sensitivity for applying to high-speed apparatuses and are required to have further improvement in repetition characteristics.

In order to achieve a high sensitivity, a function-separated photoconductor having a multilayer structure can be formed by stacking a charge-generating layer on a charge-transporting layer. The photoconductor thus formed may be used as a positive charge type, but in this type since the charge-generating layer is formed on the top surface, a stability problem is caused by corona discharge, light irradiation, and mechanical wearing when it is used repeatedly. In this respect, it is proposed to further provide a protecting layer on the charge-generating layer, but a decrease in electrical characteristics such as sensitivity cannot be overcome, though the mechanical wearing can be improved.

Additionally, there is also proposed to form a photoconductor by stacking a charge-transporting layer having electron-transporting property on a charge-generating layer.

2,4,7-Trinitro-9-fluorenone is known as a charge-transporting material having electron-transporting property, but this material is carcinogenic, which is a safety problem. Additionally, the Patent Documents suggest quinone compounds (see; Japanese Unexamined Patent Application Publication No. 1-206349, Japanese Unexamined Patent Application Publication No. 3-290666, Japanese Unexamined Patent Application Publication No. 8-278643, Japanese Unexamined Patent Application Publication No. 9-190002, Japanese Unexamined Patent Application Publication No. 9-190003, Japanese Unexamined Patent Application Publication No. 2001-222122, Japanese Unexamined Patent Application Publication No. 2003-270817, Japanese Unexamined Patent Application Publication No. 2003-270818) and, in addition to them, a large number of photoconductors containing materials having excellent electron-transporting property are proposed (see: Japanese Unexamined Patent Application Publication No. 2000-143607, Japanese Unexamined Patent Application Publication No. 2000-199979, Japanese Unexamined Patent Application Publication No. 2001-215742, Japanese Unexamined Patent Application Publication No. 2002-62673, Japanese Unexamined Patent Application Publication No. 2003-228185, Japanese Unexamined Patent Application Publication No. 2003-238561).

As described above, the charge-transporting materials having an electron-transporting property have been widely studied. However, based on a recent demand for high-sensitive photoconductors, there has been required a photoconductor achieving greater performance by using a novel charge-transporting material having a more excellent electron-transporting property.

### SUMMARY OF THE INVENTION

Accordingly, it is an object of the present invention to provide a novel compound having a superior electron-transporting property useful for electrophotographic photoconductors and organic electroluminescence (EL). Furthermore, it is an object of the present invention to provide a positive charge type electrophotographic photoconductor for high sensitive copiers or printers and an electrophotographic apparatus using the same, by using the novel organic compound in a photosensitive layer as an electron-transporting material.

In order to achieve the above-mentioned objects, the present inventors have diligently researched a variety of organic materials and have found that specific compounds represented by Formula (I) shown below have a superior electron-transporting property and that a high sensitive positive charge type photoconductor can be produced by using these compounds as a charge-transporting material. Thus, the present invention has been completed.

Namely, in order to overcome the above-mentioned problems, a novel quinone compound according to the present invention has a structure represented by Formula (I): 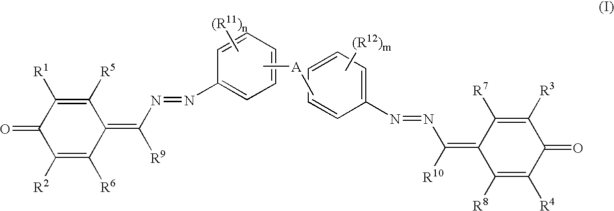
wherein R¹, R², R³, R⁴, R⁵, R⁶, R⁷ and R⁸ may be the same or different and each denotes hydrogen or a substituted or unsubstituted alkyl group having 1 to 6 carbon atoms; R⁹ and R¹⁰ may be the same or different and each denotes hydrogen, a substituted or unsubstituted alkyl group having 1 to 6 carbon atoms, a substituted or unsubstituted aryl group, or a substituted or unsubstituted heterocyclic group; R¹ and R⁵, R² and R⁶, R³ and R⁷, and R⁴ and R⁸ may bind to each other to form a ring, respectively; R¹¹ and R¹² may be the same of different and each denotes a halogen, a substituted or unsubstituted alkyl group having 1 to 6 carbon atoms, an alkoxy group having 1 to 6 carbon atoms, an alkyl halide group having 1 to 6 carbon atoms, a nitro group, a substituted or unsubstituted aryl group, or a substituted or unsubstituted heterocyclic group; n and m each denotes an integer of 0 to 4; when n is larger than 1, the plurality of R¹¹ may bind to each other to form a ring; when m is larger than 1, the plurality of R¹² may bind to each other to form a ring; A denotes oxygen or SO₂; and the substituent is a halogen, an alkyl group having 1 to 6 carbon atoms, an alkoxy group having 1 to 6 carbon atoms, an alkyl halide group having 1 to 6 carbon atoms, a nitro group, an aryl group, or a heterocyclic group.

An electrophotographic photoconductor according to the present invention includes a photosensitive layer containing a charge-generating material and a charge-transporting material on an electrically conductive substrate, wherein the photosensitive layer contains at least a compound represented by Formula (I): 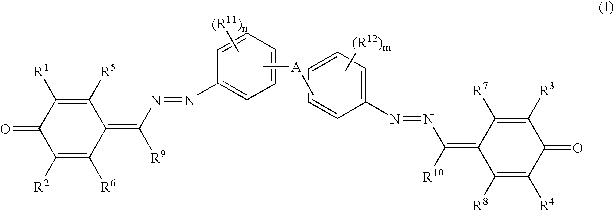
wherein R¹, R², R³, R⁴, R⁵, R⁶, R⁷ and R⁸ may be the same or different and each denotes hydrogen or a substituted or unsubstituted alkyl group having 1 to 6 carbon atoms; R⁹ and R¹⁰ may be the same or different and each denotes hydrogen, a substituted or unsubstituted alkyl group having 1 to 6 carbon atoms, a substituted or unsubstituted aryl group, or a substituted or unsubstituted heterocyclic group; R^{1 and R}⁵, R² and R⁶, R³ and R⁷, and R⁴ and R⁸ may bind to each other to form a ring, respectively; R¹¹ and R¹² may be the same or different and each denotes a halogen, a substituted or unsubstituted alkyl group having 1 to 6 carbon atoms, an alkoxy group having 1 to 6 carbon atoms, an alkyl halide group having 1 to 6 carbon atoms, a nitro group, a substituted or unsubstituted aryl group, or a substituted or unsubstituted heterocyclic group; n and m each denotes an integer of 0 to 4; when n is larger than 1, the plurality of R¹¹ may bind to each other to form a ring; when m is larger than 1, the plurality of R¹² may bind to each other to form a ring; A denotes oxygen or SO₂; and the substituent is a halogen, an alkyl group having 1 to 6 carbon atoms, an alkoxy group having 1 to 6 carbon atoms, an alkyl halide group having 1 to 6 carbon atoms, a nitro group, an aryl group, or a heterocyclic group.

In the photoconductor according to the present invention, the photosensitive layer is a single-layer photosensitive layer containing a charge-generating material, a charge-transporting material, and a resin binder. The charge-transporting material contains an electron-transporting material and a hole-transporting material. It is suitable that the electron-transporting material contains at least a compound having a structure represented by the Formula (I). In particular, the photoconductor can be suitably applied to electrophotographic apparatuses which perform a charging process by a positively charging process.

In the photosensitive layer of the photoconductor according to the present invention, known hole-transporting materials which are, for example, disclosed in Japanese Unexamined Patent Application Publication No. 2000-314969 can be used as a hole-transporting material. In particular, it is preferable that the hole-transporting material contains a styryl compound.

Furthermore, in the photosensitive layer of the photoconductor according to the present invention, known charge-generating materials can be used as a charge-generating material. In particular, it is preferable that the charge-generating material contains a phthalocyanine compound. Preferable examples of the phthalocyanine compound include, but not limited to, x-type metal-free phthalocyanine, α-type titanyl phthalocyanine, and y-type titanyl phthalocyanine disclosed in Japanese Unexamined Patent Application Publication No. 2001-228637, and titanyl phthalocyanine disclosed in Japanese Unexamined Patent Application Publication No. 2001-330972.

An electrophotographic apparatus according to the present invention includes the electrophotographic photoconductor according to the present invention and performs the charging process by a positively charging process.

According to the present invention, a compound having a superior electron-transporting property can be obtained. Electric characteristics of electronic devices can be improved by applying the compound to electrophotographic photoconductors or organic ELs of the electronic devices.

Furthermore, according to the present invention, in the electrophotographic photoconductor including a photosensitive layer on an electrically conductive substrate, the photosensitive layer contains a specific compound having such an electron-transporting property as an electron-transporting material. Therefore, the electron-transporting property can be improved and superior electric characteristics can be achieved. Additionally, since the charge-trapping is decreased, superior repetition-stability can be also achieved.

Thus, according to the present invention, high durability electrophotographic photoconductor excellent in electric characteristics and repetition stability can be obtained, and such an electrophotographic photoconductor is useful for electrophotographic apparatuses using electrophotographic systems, such as copiers, printers, and fax machines.

### BRIEF DESCRIPTION OF THE DRAWINGS

FIG. 1 is a schematic cross-sectional view illustrating a general structure of an electrophotographic photoconductor.

FIG. 2 is a schematic cross-sectional view illustrating an exemplary structure of a single-layer electrophotographic photoconductor.

FIG. 3 is a schematic cross-sectional view illustrating another exemplary structure of a single-layer electrophotographic photoconductor.

FIG. 4 is a schematic cross-sectional view illustrating an exemplary structure of a multilayer electrophotographic photoconductor.

FIG. 5 is a schematic cross-sectional view illustrating another exemplary structure of a multilayer electrophotographic photoconductor.

FIG. 6 is a schematic cross-sectional view illustrating another exemplary structure of a multilayer electrophotographic photoconductor.

### DESCRIPTION OF THE PREFERRED EMBODIMENTS

Examples of the compounds represented by the Formula (I) include, but not limited to, compounds shown by the following structural formulae (I-1) to (I-80). In the following examples, 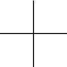
denotes a t-butyl group.

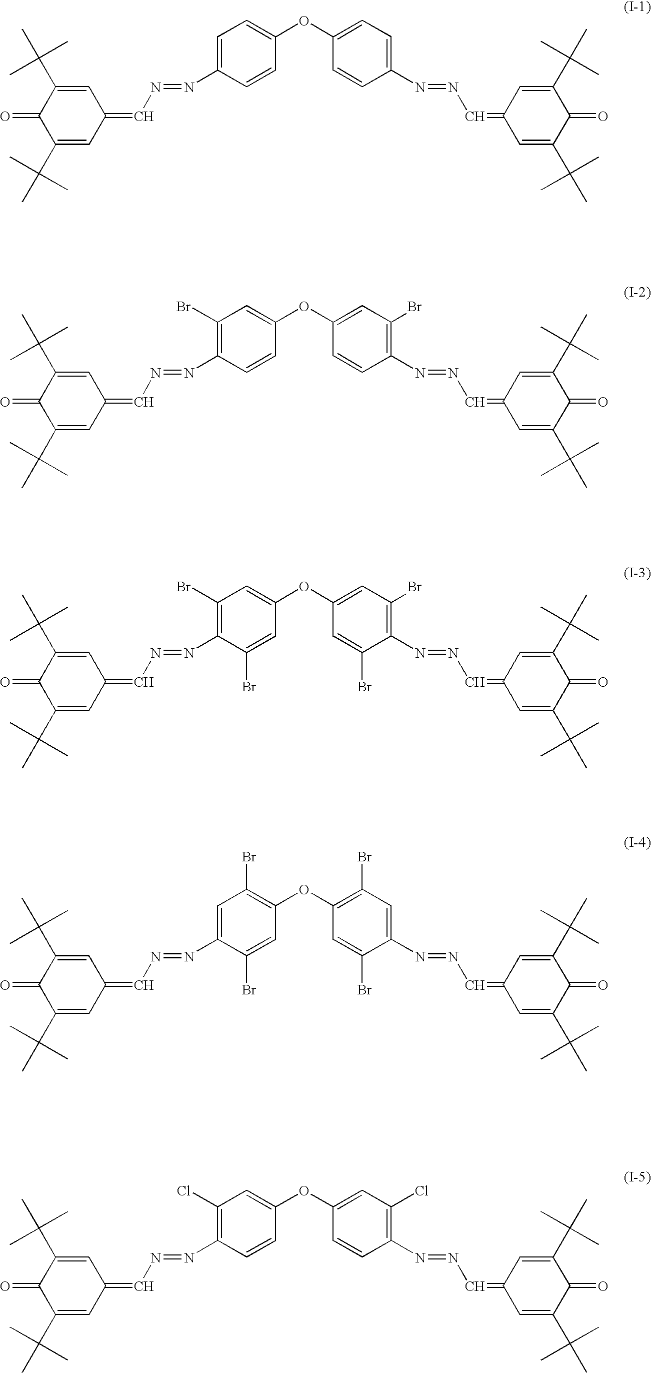

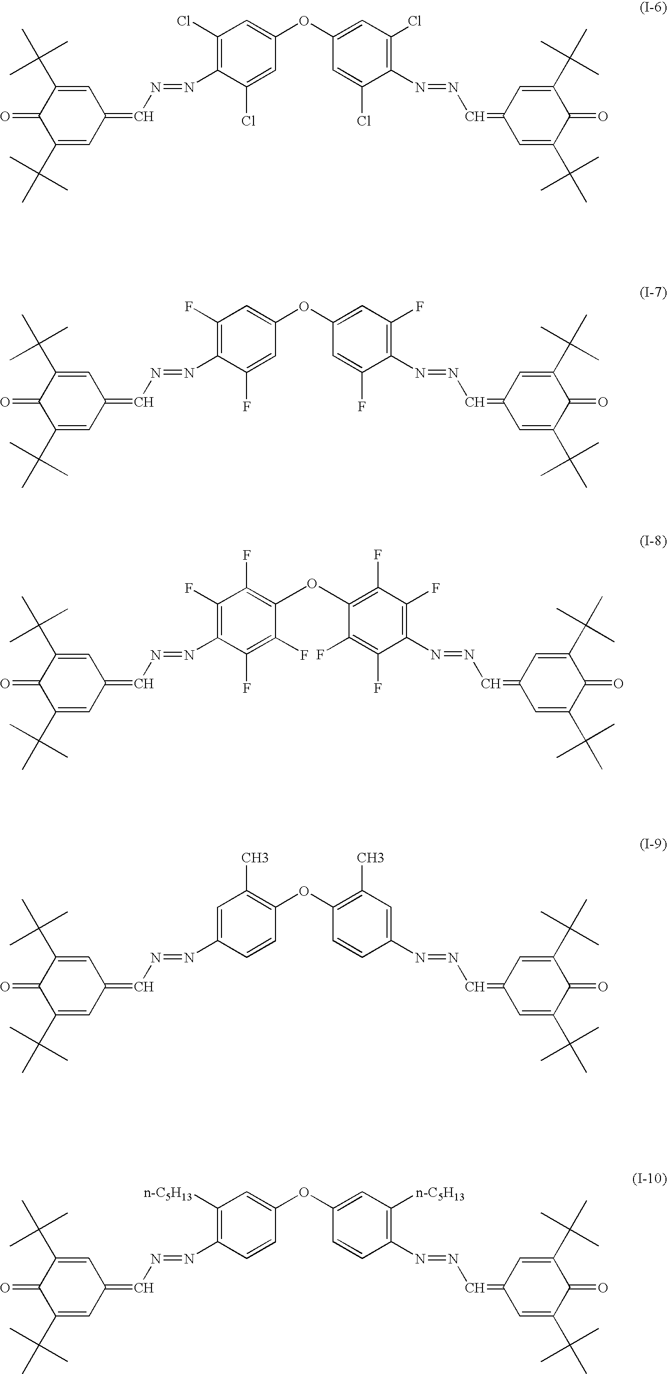

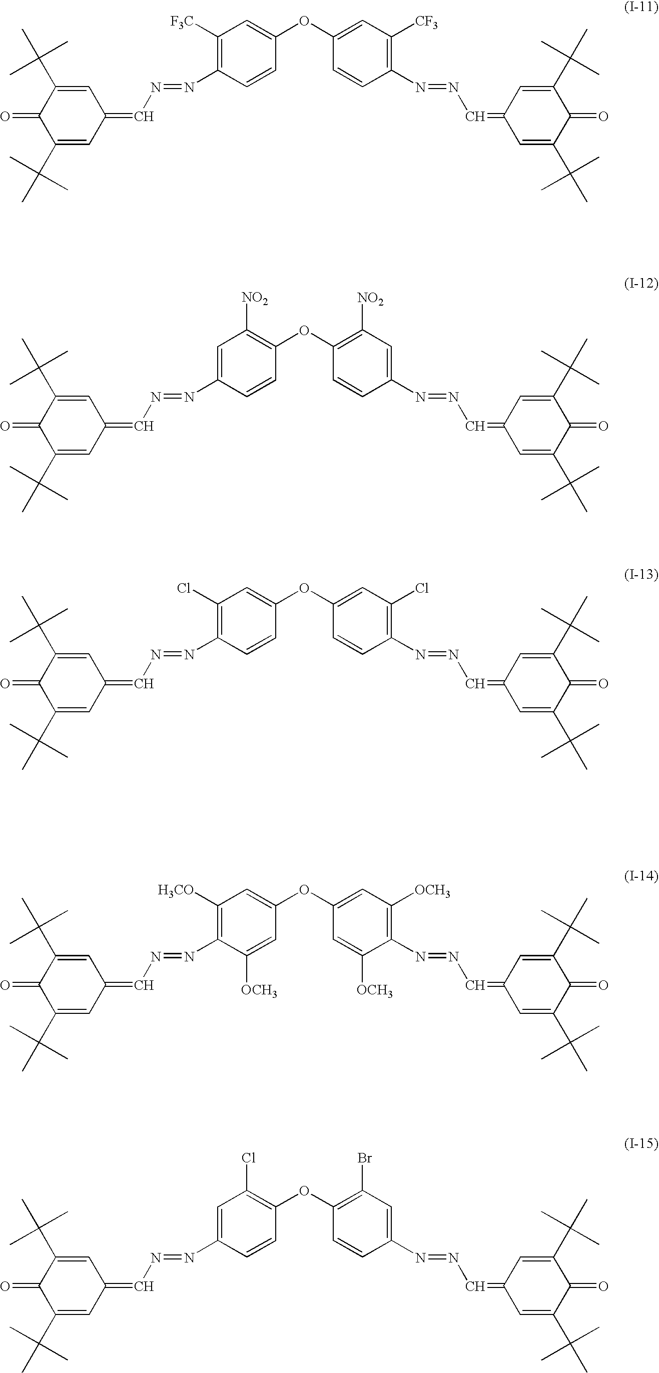

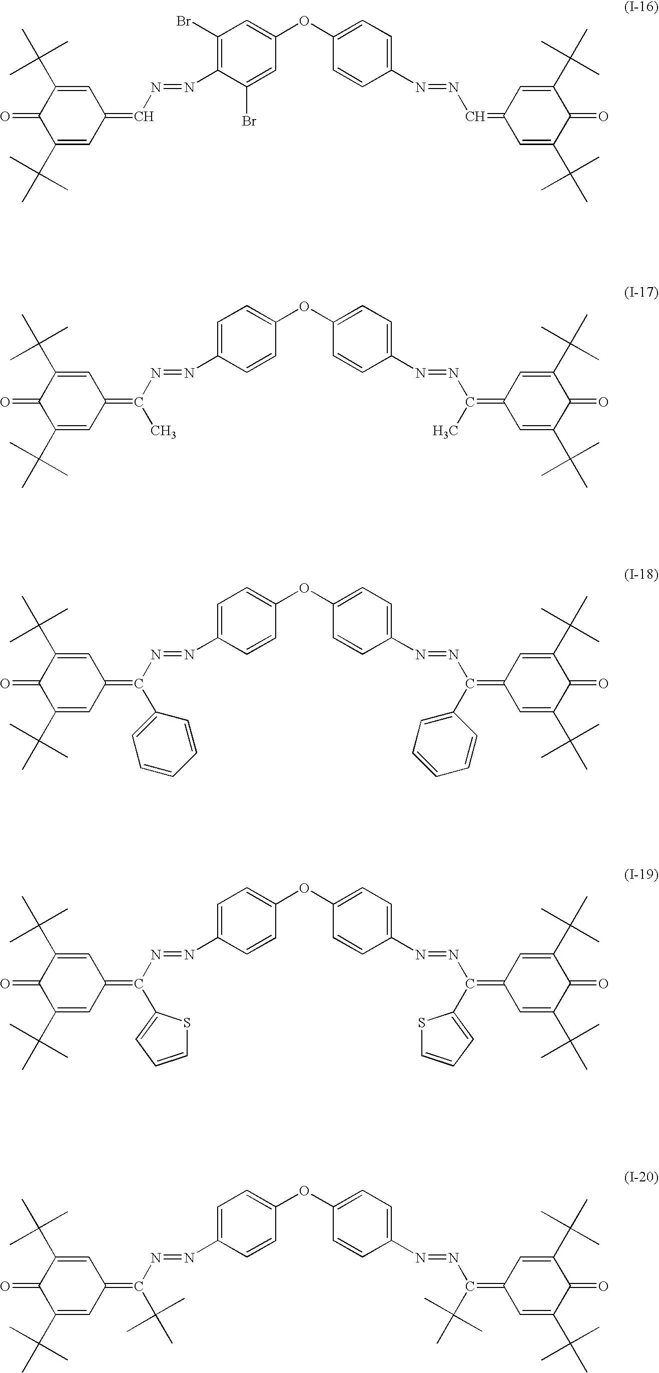

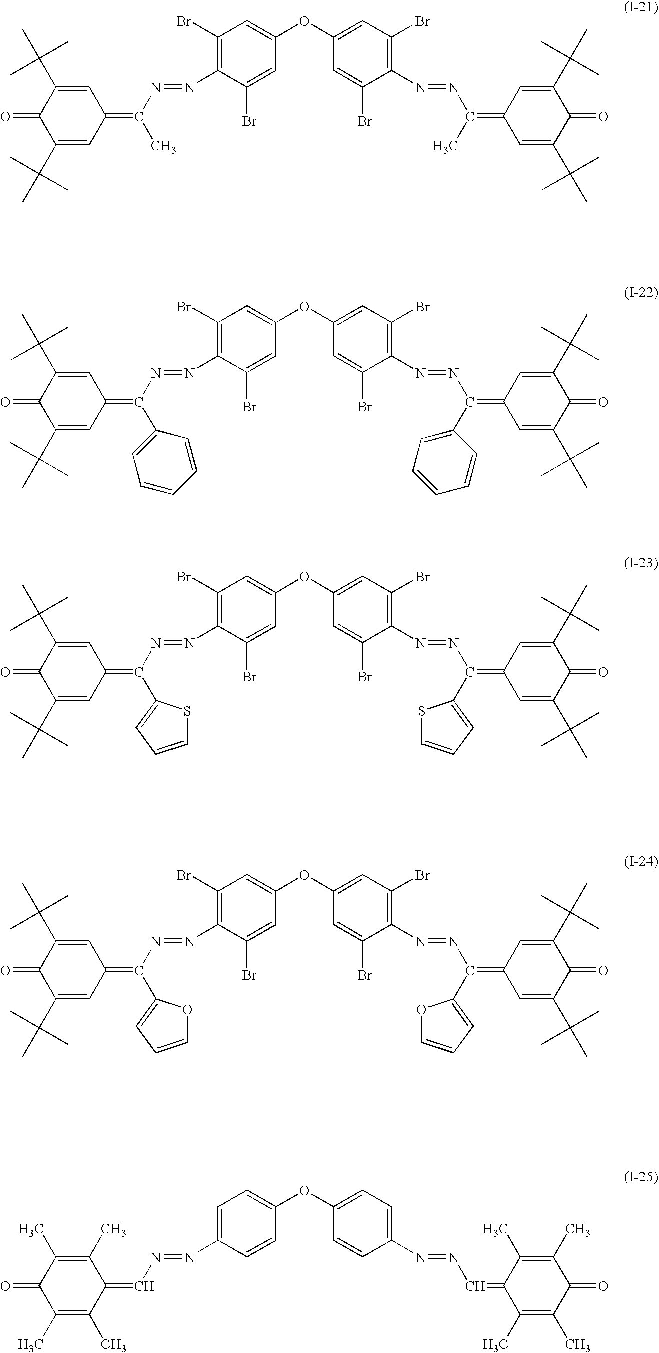

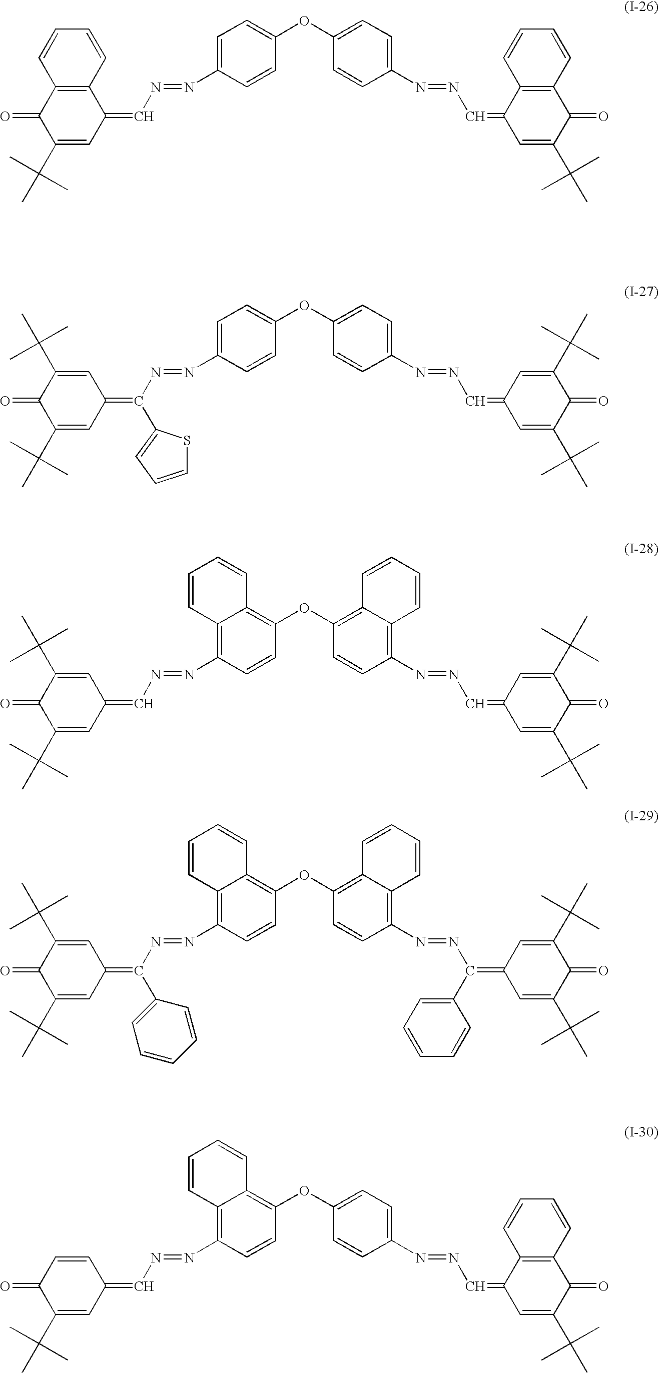

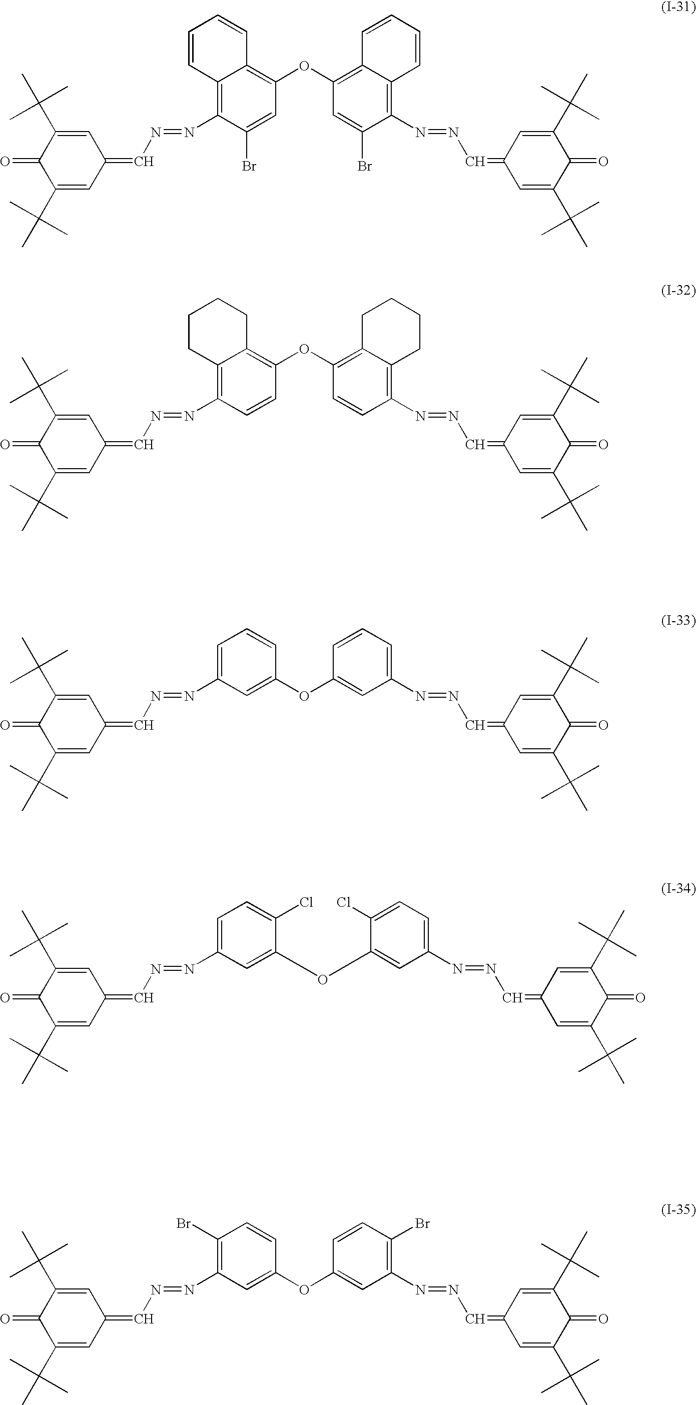

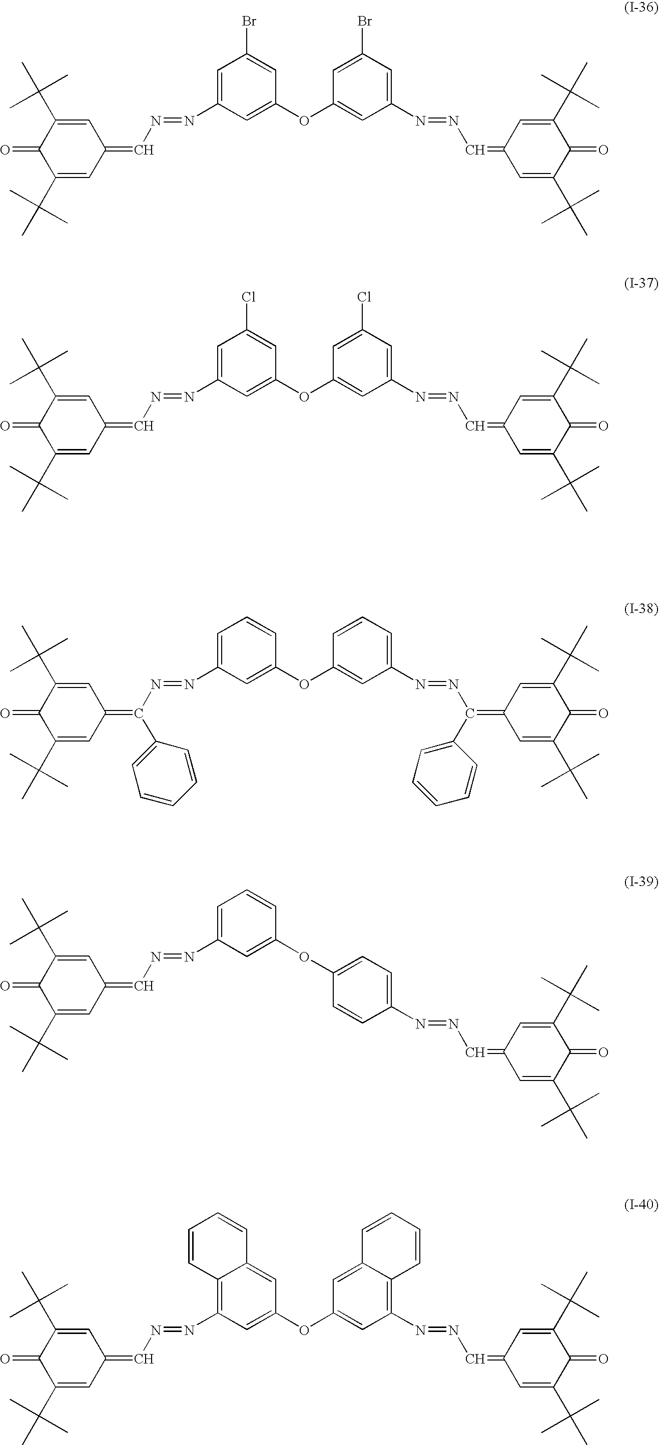

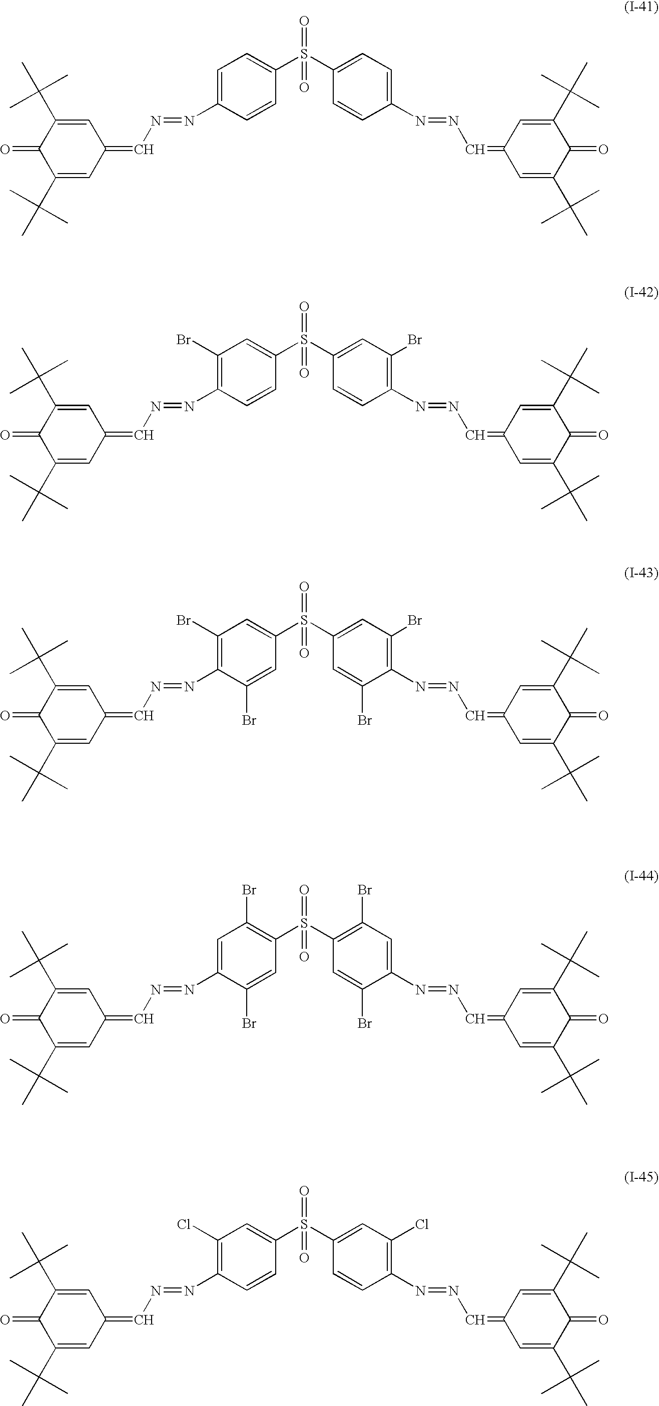

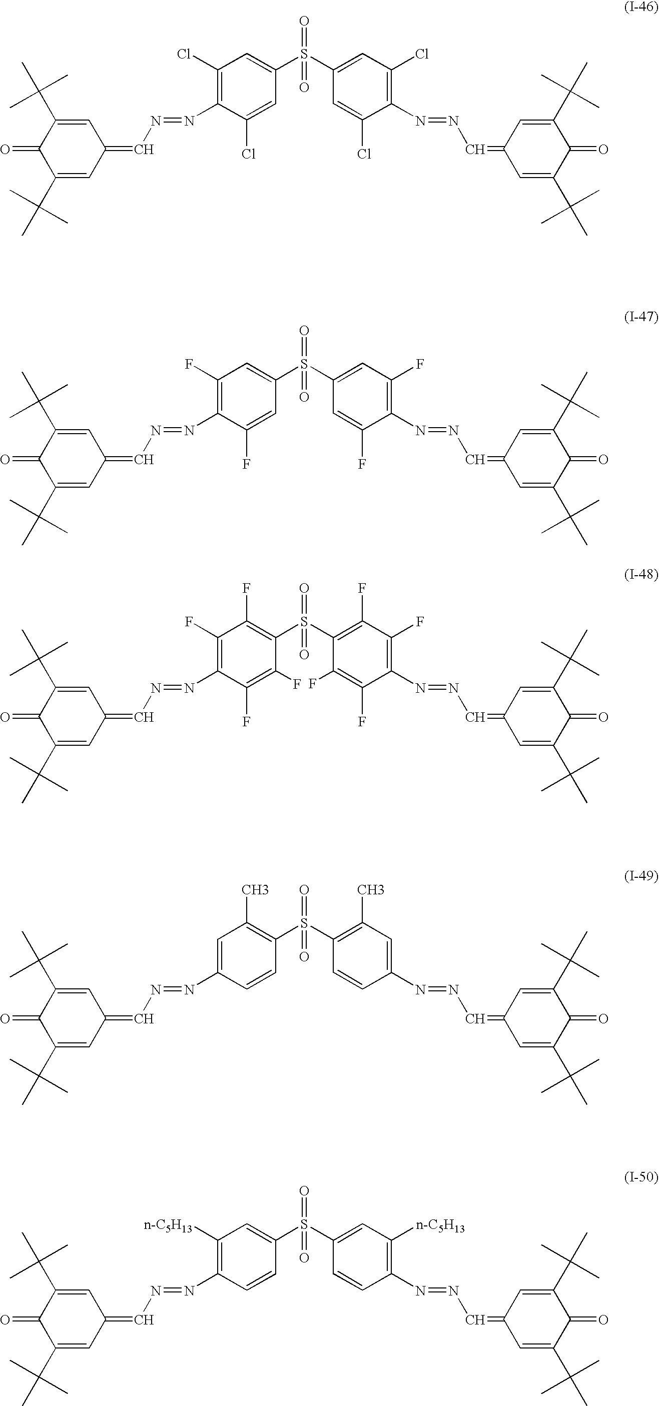

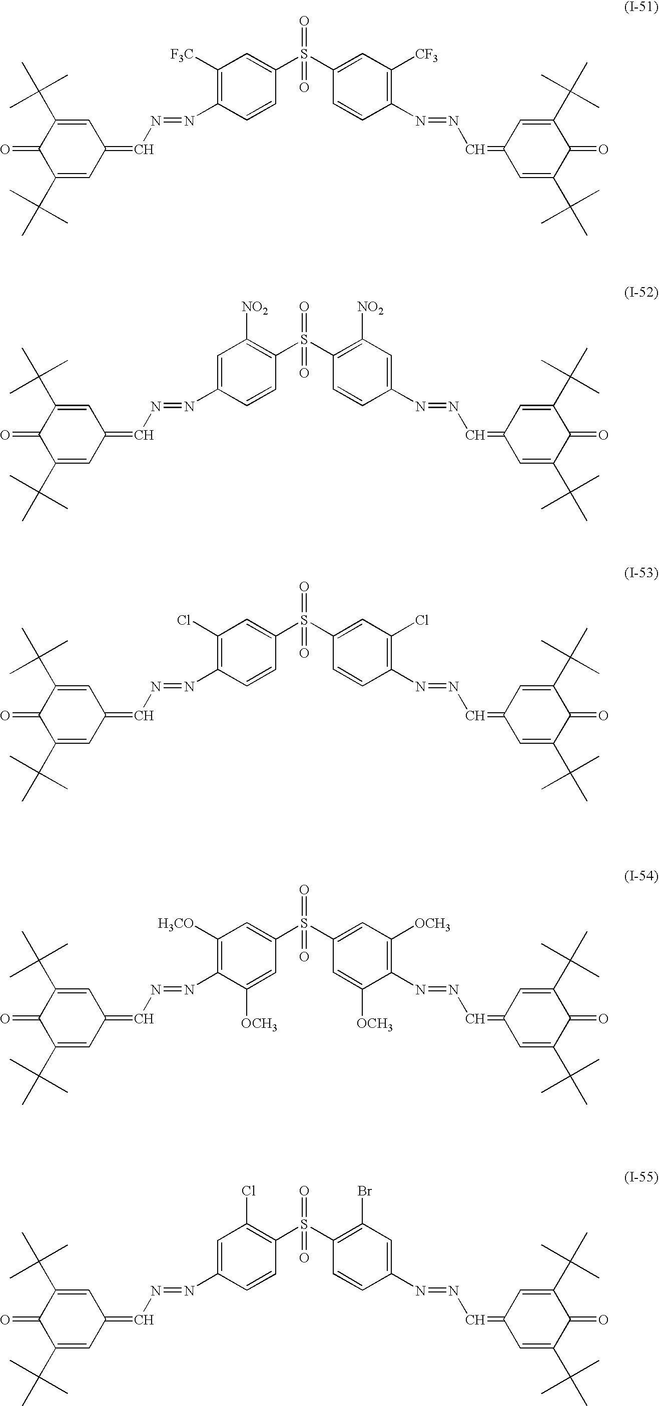

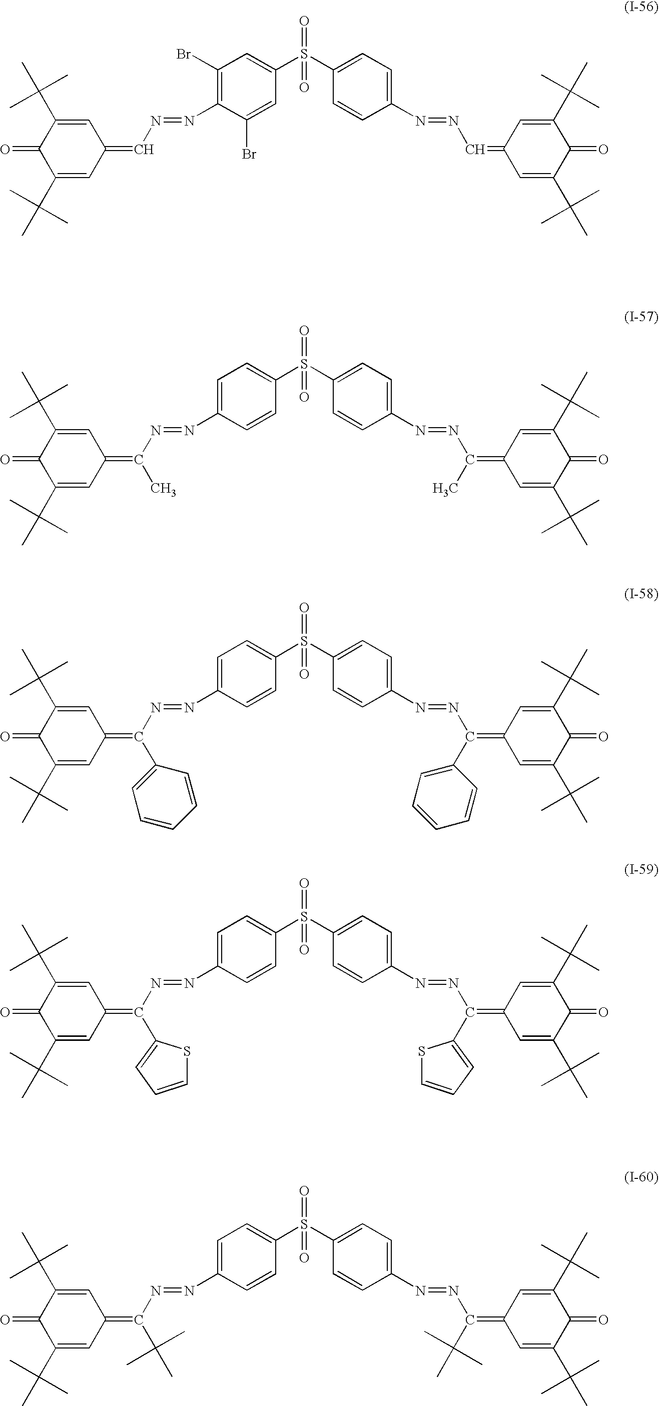

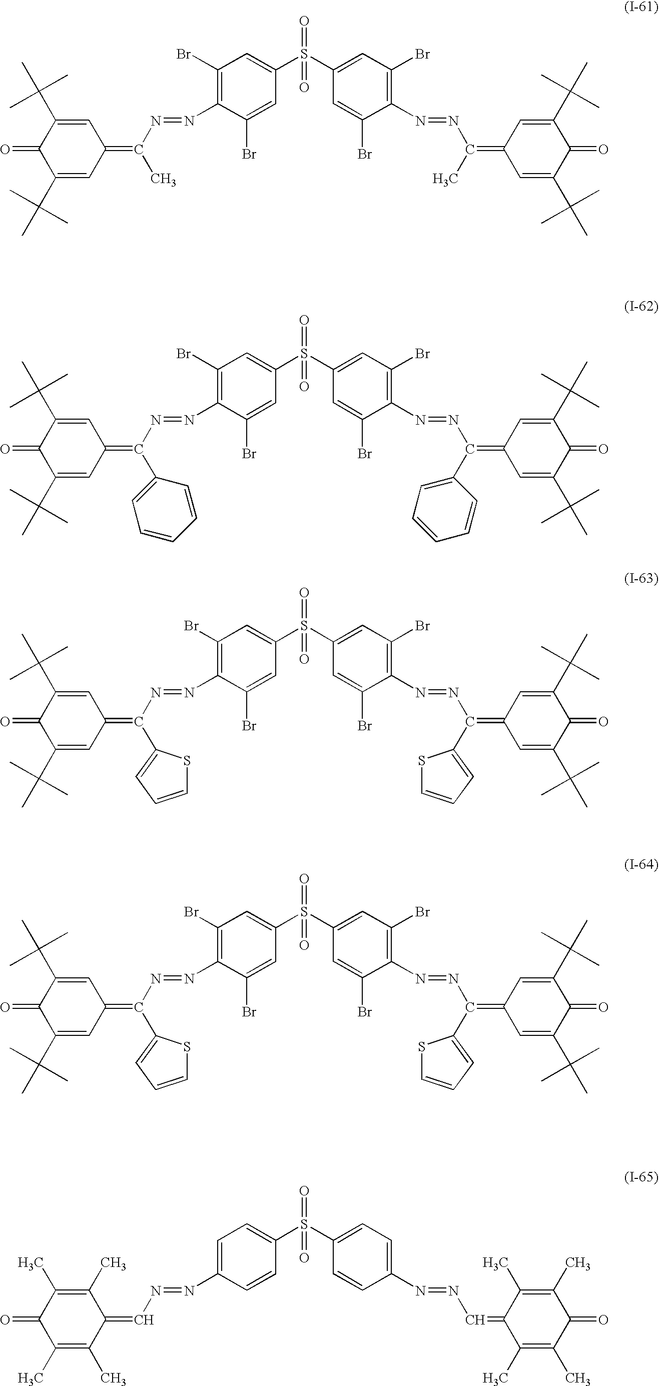

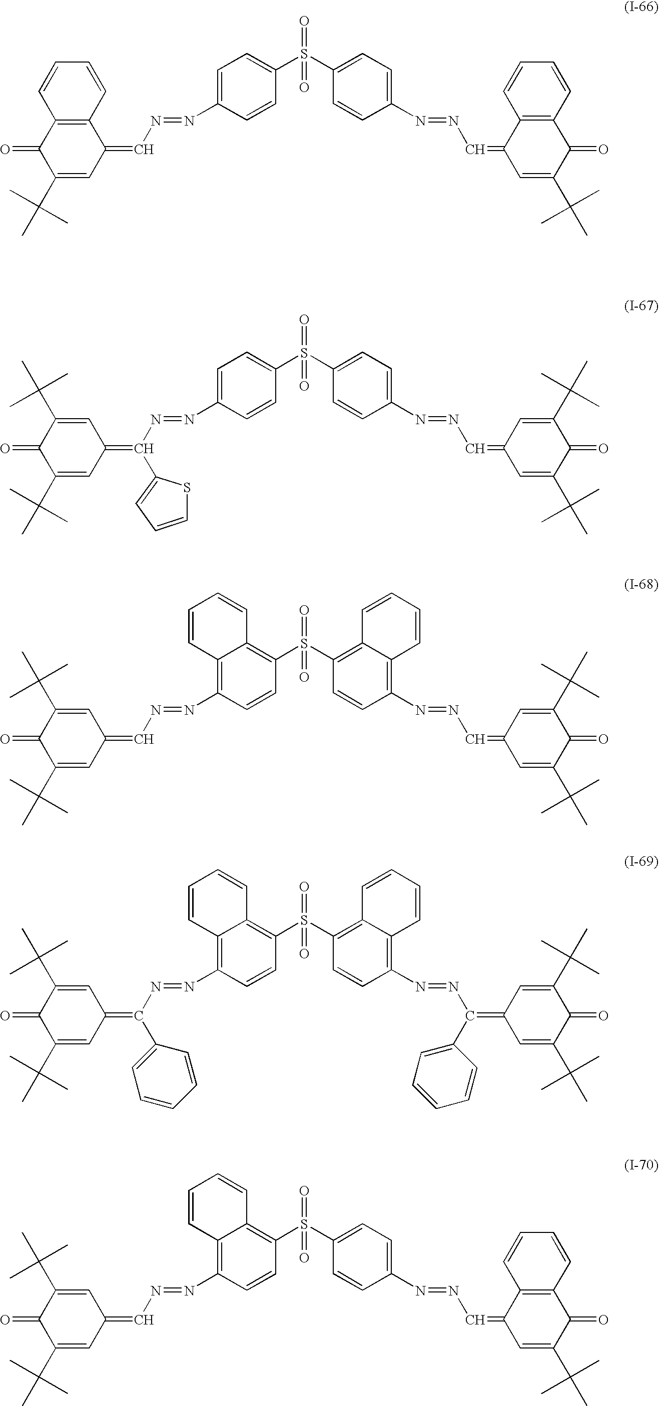

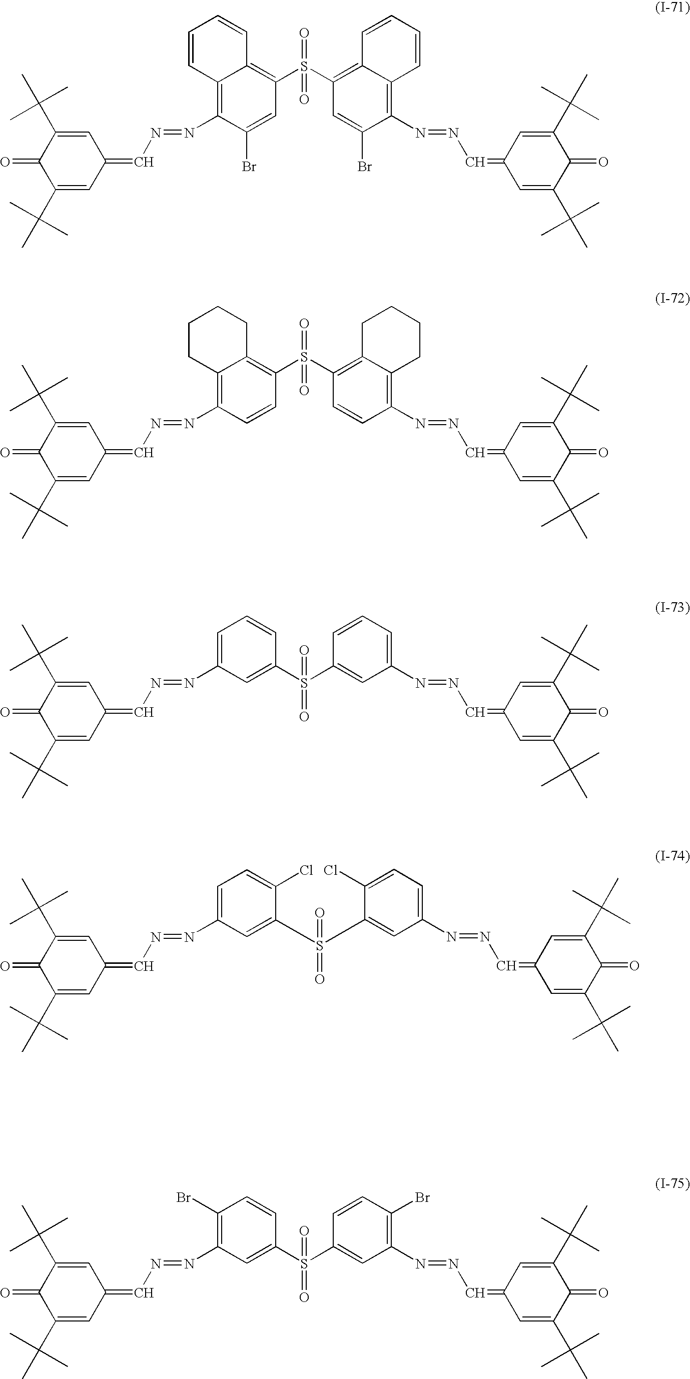

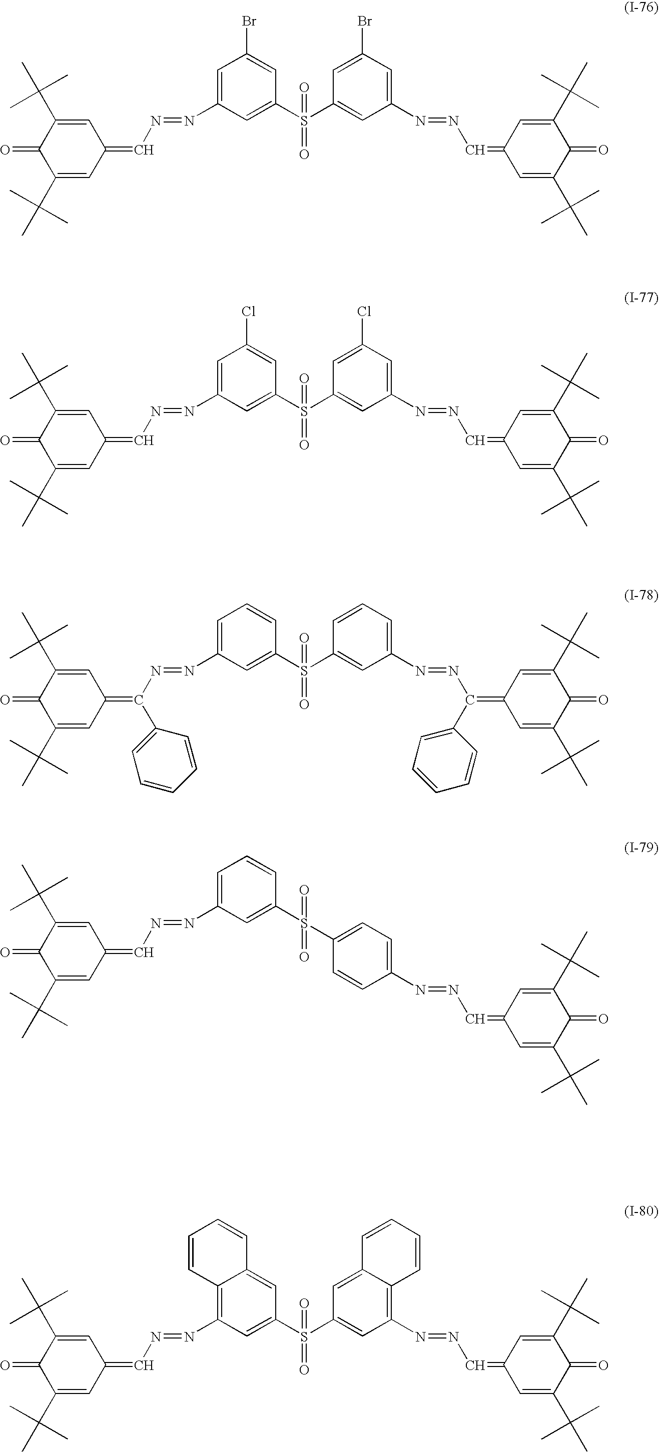

The quinone compounds of the present invention represented by the Formula (I) can be manufactured, for example, by a method shown by the following Scheme 1. In the following formulae, R¹ to R¹² and A are the same as those in the Formula (I).

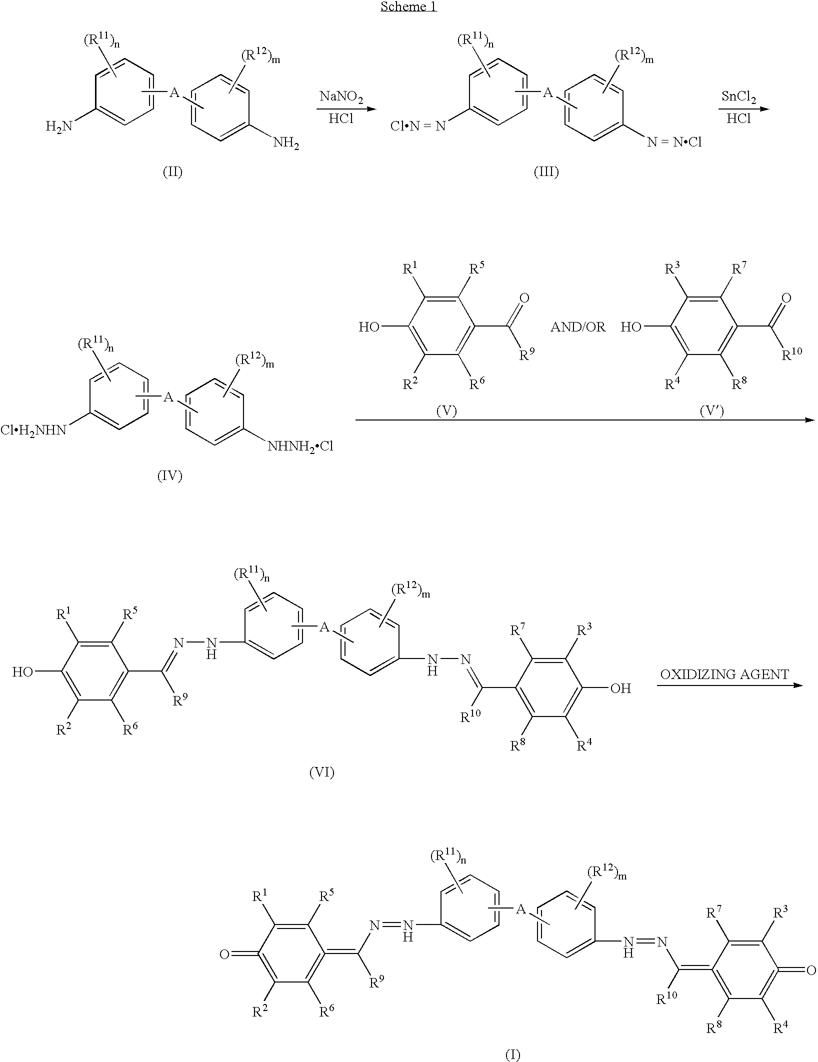

First, bisanilines (II) are converted to bisdiazonium salts (III) in hydrochloric acid by using sodium nitrite, and then converted to bishydrazine hydrochloride salts (IV) by using a reducing agent such as stannous chloride, sodium sulfite, or potassium sulfite. The resulting bishydrazine hydrochloride salts (IV) and carbonyl compounds represented by structural formulae (V) and/or (V′) are condensed by using a base such as pyridine, triethylamine, or sodium acetate to prepare bishydrazones represented by structural formula (VI). Last, quinones (represented by the Formula (I)) of interest can be synthesized by conducting a reaction of the resulting bishydrazones (VI) with an inorganic oxidizing agent such as manganese dioxide, potassium permanganate, or potassium ferricyanide or an organic oxidizing agent such as 2,3-dichloro-5,6-dicyano-1,4-benzoquinone by using a halogen solvent such as chloroform or methylene chloride or a hydrocarbon-based solvent such as benzene, toluene, or xylene in the temperature range of room temperature to a reflux temperature of the solvent.

Since the quinone compounds of the present invention represented by the Formula (I) have a superior electron-transporting property, they are useful as a so-called electron-transporting material, in particular, they can be suitably used as a material for a photosensitive layer of electrophotographic photoconductors and a material for a functional layer, such as an electron-transporting layer, of organic ELs.

Specific embodiments of the electrophotographic photoconductor of the present invention will now be described in detail with reference to the drawings.

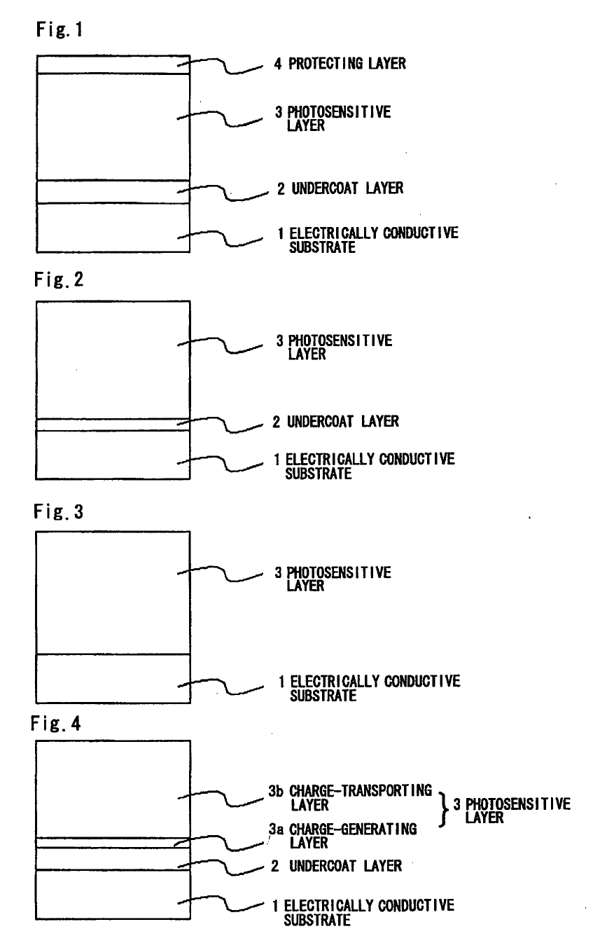

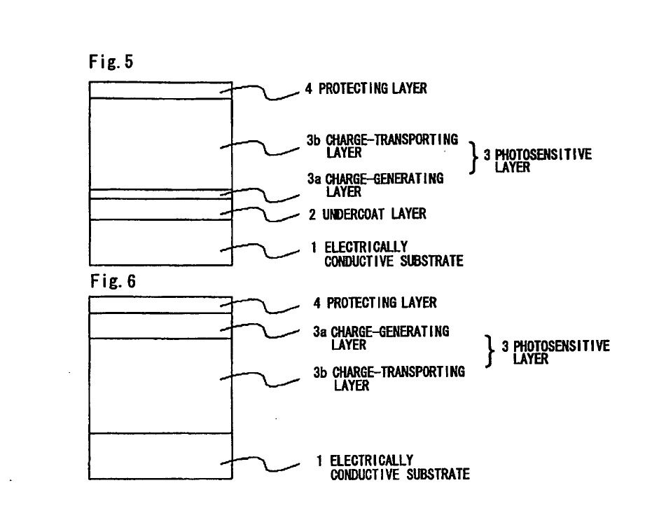

FIG. 1 is a schematic cross-sectional view illustrating a photoconductor according to an embodiment of the present invention. Reference numeral 1 indicates an electrically conductive substrate, reference numeral 2 indicates an undercoat layer, reference numeral 3 indicates a photosensitive layer, and reference numeral 4 indicates a protecting layer. The undercoat layer 2 and the protecting layer 4 may be provided if necessary. The photosensitive layer 3 may be a single-layer type, namely, a single layer includes both a charge-generating function and a charge-transporting function, or the photosensitive layer 3 may be a function-separated type, namely, a charge-generating layer and a charge-transporting layer are stacked. The photoconductors according to the main embodiment have layer structures shown in FIGS. 2 to 6. FIGS. 2 and 3 show single-layer photoconductors of which the photosensitive layer 3 is a single layer. FIGS. 4 and 5 show function-separated multilayer photoconductors of which the photosensitive layer 3 is formed by stacking a charge-generating layer 3a and a charge-transporting layer 3b in this order on the undercoat layer 2. FIG. 6 shows a function-separated multilayer photoconductor of which the photosensitive layer 3 is formed by stacking a charge-transporting layer 3b and a charge-generating layer 3a in this order and further stacking the protecting layer 4 on the charge-generating layer 3a. However, the present invention is not limited to these photoconductors having the layer structures shown by the drawings.

The electrically conductive substrate 1 serves as both an electrode of the photoconductor and a supporting member for other layers, and may be a cylindrical, plate-like, or film-like shape. The electrically conductive substrate 1 may be a conductively treated metal, such as aluminum, stainless steal, and nickel, glass, or resins.

The undercoat layer 2 may be provided, if necessary, for preventing injection of unnecessary charge from the electrically conductive substrate to the photosensitive layer, coating a defect in the substrate surface, and improving an adhesive property of the photosensitive layer, and is a layer mainly made of a resin or an oxidized film of alumite or the like. The resin binders for the undercoat layer may be polycarbonate resins, polyester resins, polyvinyl acetal resins, polyvinyl butyral resins, vinyl chloride resins, vinyl acetate resins, polyethylenes, polypropylenes, polystyrenes, acrylic resins, polyurethane resins, epoxy resins, melamine resins, phenol resins, silicone resins, polyamide resins, polystyrene resins, polyacetal resins, polyarylate resins, polysulfone resins, and polymers of methacrylic acid ester. These compounds may be used alone or in a combination including copolymers of these compounds. The resin binders may contain one or more types of microparticles of a metal oxide such as silicon oxide (silica), titanium oxide, zinc oxide, calcium oxide, aluminum oxide (alumina), and zirconium oxide; of a metal sulfide such as barium sulfide and calcium sulfide; or of a metal nitride such as silicon nitride and aluminum nitride. The surfaces of these microparticles may be treated with a silane coupling agent or may be coated with a metal oxide film.

The thickness of the undercoat layer depends on its composition, but can be arbitrarily determined in the range of that adverse effects, such as an increase in residual potential, do not occur even if it is used repeatedly. In general, the thickness is about 0.01 to 50 μm. The undercoat layer may be a lamination of a plurality of layers.

The photosensitive layer 3 is mainly composed of a charge-generating layer 3a and a charge-transporting layer 3b when it is a function-separated type, but it is composed of a single layer when it is a single-layer type. However, a plurality of layers having a similar function may be stacked.

The charge-generating layer 3a is formed by vacuum deposition of an inorganic or organic photoconductive material or by application of inorganic or organic photoconductive material particles dispersed in a resin binder. The charge-generating layer generates charges by absorbing light. It is important that not only the charge-generating efficiency but also the injection efficiency of the generated charges to the charge-transporting layer 3b are high. The electric-field dependency is desirable low so that the injection is well performed even if the electric field is low.

Since the charge-generating layer only needs to provide a charge-generating function, the thickness of the charge-generating layer may be determined on the basis of a light absorption coefficient of the charge-generating material. Generally, the thickness is 0.1 to 50 μm. When the multilayer photoconductor has a charge-transporting layer on the charge-generating layer, the thickness is generally 5 μm or less, preferably, 1 μm or less.

The charge-generating layer is almost exclusively composed of a charge-generating material and may further contain a charge-transporting additive. Examples of the charge-generating material include phthalocyanine-type pigments, azo pigments, anthanthrone pigments, perylene pigments, perynone pigments, squarylium pigments, thiapyrylium pigments, quinacridone pigments, and combinations thereof. In particular, disazo pigments and trisazo pigments of the azo pigments, N,N′-bis(3,5-dimethylphenyl)-3,4:9,10-perylenebis(carboximide) of the perylene pigments, and metal-free phthalocyanine, copper phthalocyanine, and titanyl phthalocyanine of the phthalocyanine-type pigments are preferable.

In the present invention, among these charge-generating materials, the phthalocyanine-type pigments are most preferable. Such phthalocyanines exist in various crystal forms and are known as an x-type metal-free phthalocyanine, a τ-type metal-free phthalocyanine, an ε-type copper phthalocyanine, an α-type titanyl phthalocyanine, a β-type titanyl phthalocyanine, a y-type titanyl phthalocyanine, an amorphous titanyl phthalocyanine, and a titanyl phthalocyanine that shows the maximum peak at 9.6° of Bragg angle 2θ in a CuKα X-ray diffraction spectrum as disclosed in Japanese Unexamined Patent Application Publication No. 8-209023. Among them, for example, the x-type metal-free phthalocyanine, α-type titanyl phthalocyanine, and y-type titanyl phthalocyanine that are disclosed in Japanese Unexamined Patent Application Publication No. 2001-228637, and the titanyl phthalocyanine that is disclosed in Japanese Unexamined Patent Application Publication No. 2001-330972 are more preferable.

Some of the above-mentioned charge-generating materials have a charge-transporting function in addition to the charge-generating function. In particular, since the azo pigments and the perylene pigments have the charge-transporting function, it can be used as a charge-transporting material in addition to the purpose of generating charges.

The resin binder for the charge-generating layer may be polyvinyl acetal resins, polyvinyl butyral resins, vinyl chloride resins, vinyl acetate resins, silicone resins, polycarbonate resins, polyester resins, polyethylenes, polypropylenes, polystyrenes, acrylic resins, polyurethane resins, epoxy resins, melamine resins, polyamide resins, polystyrene resins, polyacetal resins, polyarylate resins, polysulfone resins, and polymers of methacrylic acid ester. These compounds may be used alone or in an appropriate combination including copolymers of these compounds. Mixtures of the same type of resins with different molecular weights may be used. The content of the resin binder is preferably 10 to 90 wt %, more preferably 20 to 80 wt % with respect to the solid components of the charge-generating layer.

Here, when a charge-transporting material is added to the charge-generating layer, charge-transporting materials used for the charge-transporting layer, which will be described below, can be used as the charge-transporting material. Additionally, the compounds represented by the Formula (I) according to the present invention can be used. The content of the charge-transporting material added to the charge-generating layer is about 0.1 to 50 wt % with respect to the solid components of the charge-generating layer.

The charge-transporting layer 3b is a film that is prepared by dispersing a charge-transporting material in a resin binder. The charge-transporting layer 3b retains charges of a photoconductor in the dark as an insulating layer and transports charges injected from the charge-generating layer in light-absorbing.

Hole-transporting materials and electron-transporting materials are known as the charge-transporting materials. In the present invention, at least a compound represented by the Formula (I) must be used as the electron-transporting material, but other electron-transporting materials and hole-transporting materials may be simultaneously used with such a compound. The content of the charge-transporting material is preferably 10 to 90 wt %, more preferably 20 to 80 wt % with respect to the solid components of the charge-transporting layer. The compounds represented by the Formula (I) according to the present invention can exhibit the advantageous effects of the present invention when they are merely contained in the charge-transporting layer, and the content is preferably 10 to 60 wt %, more preferably 15 to 50 wt % with respect to the solid components of the charge-transporting layer.

Other known electron-transporting materials and electron-accepting materials such as succinic anhydride, maleic anhydride, dibromosuccinic anhydride, phthalic anhydride, 3-nitrophthalic anhydride, 4-nitrophthalic anhydride, pyromellitic anhydride, pyromellitic acid, trimellitic acid, trimellitic anhydride, phthalimide, 4-nitrophthalimide, tetracyanoethylene, tetracyanoquinodimethane, chloranil, bromanil, o-nitrobenzoic acid, trinitrofluorenone, quinone, benzoquinone, diphenoquinone, naphthoquinone, anthraquinone, and stilbenequinone can be used. In particular, compounds, which are disclosed in Japanese Unexamined Patent Application Publication No. 2000-314969, represented by structural formulae (ET1-1) to (ET1-16), (ET2-1) to (ET2-16), (ET3-1) to (ET3-12), (ET4-1) to (ET4-32), (ET5-1) to (ET5-8), (ET6-1) to (ET6-50), (ET7-1) to (ET7-14), (ET8-1) to (ET8-6), (ET9-1) to (ET9-4), (ET10-1) to (ET10-32), (ET11-1) to (ET11-16), (ET12-1) to (ET12-16), (ET13-1) to (ET13-16), (ET14-1) to (ET14-16), (ET15-1) to (ET15-16), and (ET-1) to (ET-42) are preferable. These electron-accepting materials and electron-transporting materials can be used alone or in a combination of two or more materials.

Any hole-transporting material can be used, but styryl compounds are preferable. The styryl compounds used in the present invention include a structure represented by the following formula: 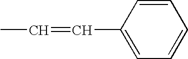
(wherein hydrogen atoms may be each substituted by a substituent).

Specific structures of the styryl compounds are represented by, for example, structural formulae (HT1-1) to (HT1-136) and (HT2-1) to (HT2-70) disclosed in Japanese Unexamined Patent Application Publication No. 2000-314969, structural formulae (V-40) to (V-57) disclosed in Japanese Unexamined Patent Application Publication No. 2000-204083, and structural formulae (HT1-1) to (HT1-70) disclosed in Japanese Unexamined Patent Application Publication No. 2000-314970, but the present invention is not limited to these compounds.

Other hole-transporting materials may be hydrazone compounds, pyrazoline compounds, pyrazolone compounds, oxadiazole compounds, oxazole compounds, arylamine compounds, benzidine compounds, stylbene compounds, polyvinylcarbazoles, and polysilanes (specific structures disclosed are shown, for example, in Japanese Unexamined Patent Application Publication No. 2000-314969, by structural formulae (HT3-1) to (HT3-39), (HT4-1) to (HT4-20), (HT5-1) to (HT5-10), and (HT-1) to (HT-37)). These hole-transporting materials can be used alone or in a combination of two or more materials.

The resin binders for the charge-transporting layer may be polycarbonate resins, polyester resins, polyvinyl acetal resins, polyvinyl butyral resins, vinyl chloride resins, vinyl acetate resins, polyethylenes, polypropylenes, polystyrenes, acrylic resins, polyurethane resins, epoxy resins, melamine resins, phenol resins, silicon-based resins, silicone resins, polyamide resins, polystyrene resins, polyacetal resins, polyarylate resins, polysulfone resins, and polymers of methacrylic acid ester. These compounds may be used alone or in a combination including copolymers of these compounds. In particular, polycarbonates including a structural unit shown by structural formulae (BD1-1) to (BD1-16) disclosed in Japanese Unexamined Patent Application Publication No. 2000-314969 as a main repeating unit are preferable. Additionally, polycarbonate resins including one or more structural units shown by structural formulae (BD-1) to (BD-7) disclosed in Japanese Unexamined Patent Application Publication No. 2000-314969 as a main repeating unit, and polyester resins are preferable. These resins can be used alone or in a combination of two or more resins. Furthermore, mixtures of the same type of resins with different molecular weights may be used. The content of the resin binder is preferably 10 to 90 wt %, more preferably 20 to 80 wt % with respect to the solid components of the charge-transporting layer.

The thickness of the charging-transporting layer is preferably 3 to 100 μm, more preferably 10 to 50 μm, for retaining the practically effective surface potential.

Generally, in the function-separated multilayer photoconductors, a charge-transporting layer is stacked on a charge-generating layer, but a charge-generating layer may be stacked on a charge-transporting layer (FIG. 6).

In the photosensitive layer of a single-layer type, a charge-generating material, a charge-transporting material, and a resin binder are used as main components. Hole-transporting materials and electron-transporting materials are used as the charge-transporting materials. In the present invention, at least a compound represented by the Formula (I) must be used as the electron-transporting material, but other charge-transporting materials (the electron-transporting materials and the hole-transporting materials) may be simultaneously used with such a compound, as in the case of the charge-transporting layer 3b. Desirably, a hole-transporting material is simultaneously used. As the charge-generating materials, the compounds used in the charge-generating layer 3a can be used. As the resin binder, the resin binders used in the charge-transporting layer 3b and the charge-generating layer 3a can be used.

The content of the charge-generating material is preferably 0.01 to 50 wt %, more preferably 0.1 to 20 wt % particularly more preferably 0.5 to 10 wt wt % with respect to the solid components of the single-layer photosensitive layer. The content of the charge-transporting material is preferably 10 to 90 wt %, more preferably 20 to 80 wt % with respect to the solid components of the single-layer photosensitive layer. The compounds represented by the Formula (I) according to the present invention can exhibit the advantageous effects of the present invention when they are merely contained in the single-layer photosensitive layer, and the content is preferably 10 to 60 wt %, more preferably 15 to 50 wt % with respect to the solid components of the single-layer photosensitive layer. The content of the hole-transporting material to be simultaneously used is preferably 10 to 60 wt %, more preferably 20 to 50 wt % with respect to the solid components of the single-layer photosensitive layer. The content of the resin binder is generally 10 to 90 wt %, preferably 20 to 80 wt % with respect to the solid components of the single-layer photosensitive layer.

The thickness of the single-layer photosensitive layer is preferably 3 to 100 μm, more preferably 10 to 50 μm, for retaining practically effective surface potential.

These photosensitive layers may contain a degradation-preventing agent such as antioxidants and photostabilizers for improving environment resistance and stability to hazardous light. Examples of compounds to be used for achieving these objects include chromanol derivatives such as tocopherol, esterified compounds, poly(aryl alkane) compounds, hydroquinone derivatives, etherified compounds, dietherified compounds, benzophenone derivatives, benzotriazole derivatives, thioether compounds, phenylenediamine derivatives, phosphonic esters, phosphates, phenol compounds, hindered phenol compounds, straight chain amine compounds, cyclic amine compounds, and hindered amine compounds.

The photosensitive layer may contain a leveling agent such as silicone oils and fluorine-based oils for the purpose of improving the leveling quality of the formed film and providing lubrication.

The photosensitive layer may further contain, for the purpose of reducing the friction coefficient or providing lubricity, microparticles of a metal oxide such as silicon oxide (silica), titanium oxide, zinc oxide, calcium oxide, aluminum oxide (alumina), and zirconium oxide, of a metal sulfate such as barium sulfate and calcium sulfate, or of a metal nitride such as silicon nitride and aluminum nitride; microparticles of a fluorine-based resin such as ethylene tetrafluoride resin or a silicone resin; or a polymer containing fluorine such as comb-type fluorine-containing graft polymer resins or a polymer containing silicon.

Additionally, the photosensitive layer may contain other known additives if necessary, as long as no substantial deterioration occurs in electrophotographic quality.

The protecting layer 4 may be provided for improving printing durability if necessary. The protecting layer may be made of a layer including a resin binder as a main component; an inorganic film that is formed by a vapor growth of amorphous carbon, amorphous silicon-carbon, or the like; or a coating film that is formed by evaporating silica, alumina, or the like. The resin binder may be those used in the charge-transporting layer 3b or three-dimensional cross-linking resins such as siloxane resins. The resin binder may further contain, for the purpose of improving the electrical conductivity, reducing the friction coefficient, or providing lubricity, microparticles of a metal oxide such as silicon oxide (silica), titanium oxide, zinc oxide, calcium oxide, aluminum oxide (alumina), and zirconium oxide, of a metal sulfate such as barium sulfate and calcium sulfate, or of a metal nitride such as silicon nitride and aluminum nitride; microparticles of a fluorine-based resin such as ethylene tetrafluoride resin or a silicone resin; or a polymer containing fluorine such as comb-type fluorine-containing graft polymer resins or a silicon-containing polymer having a network structure.

The protecting layer may further contain, for the purpose of providing charge-transporting property, the charge-transporting materials used for the photosensitive layer, electron-accepting materials, electron-transporting materials, or compounds represented by the Formula (I). Additionally, the protecting layer may contain, for the purpose of improving the leveling quality or providing lubricity, a leveling agent such as silicone oils and fluorine-based oils.

Generally, the thickness of the protecting layer is preferably in the range of 0.1 to 50 μm, more preferably 1 to 10 μm, as long as no substantial deterioration occurs in the photosensitive layer functions. The protecting layer may be a multilayer type.

A method for preparing the photoconductor of the present invention will now be described in detail. A more detailed description is available, for example, in Denshi Shashin Gakkaishi (Electrophotography), Vol. 28, No. 2, pp. 186-195, 1989, “OPC Kankotai no Seisan Gizyutsu (The manufacturing technology of OPC photoconductor)”.

When the undercoat layer 2, the photosensitive layer 3 (the charge-transporting layer 3a and the charge-transporting layer 3b), and the protecting layer 4 are formed by coating, they can be formed by preparing a coating solution by dissolving and dispersing the constituting materials in an appropriate solvent, applying the coating solution by an appropriate application method, and removing the solvent by drying.

Principal examples of the solvent include alcohols such as methanol, ethanol, n-propanol, i-propanol, n-butanol, and benzyl alcohol; ketones such as acetone, methylethylketone (MEK), methylisobutylketone, and cyclohexanone; amides such as dimethylformamide (DMF) and dimethylacetamide; sulfoxides such as dimethylsulfoxide; cyclic or linear ethers such as tetrahydrofuran (THF), dioxane, dioxolane, diethylether, methyl cellosolve, and ethyl cellosolve; esters such as methyl acetate, ethyl acetate, and n-butyl acetate; aliphatic hydrocarbon halides such as methylene chloride, chloroform, carbon tetrachloride, dichloroethylene, and trichloroethylene; mineral oils such as ligroin; aromatic hydrocarbons such as benzene, toluene, and xylene; and aromatic hydrocarbon halides such as chlorobenzene and dichlorobenzene. These solvents may be used alone or in a combination of two or more solvents.

The dissolving and dispersing of the coating solution can be principally conducted by known methods, for example, a method using a paint shaker (paint conditioner), a ball mill, or a bead mill (sand grinder) such as Dyno-mill, and ultrasonic dispersion. The application of the coating solution can be principally conducted by known methods, for example, dip coating, ring coating (seal coating), spray coating, bar coating, and blade coating.

The temperature and the time for the drying may be adequately determined on the basis of the type of solvent and manufacturing cost. Preferably, the temperature for the drying is in the range of room temperature to 200° C. and the time for the drying is in the range of 10 min to 2 hr. More preferably, the temperature for the drying is in the range of the boiling temperature of the solvent to the boiling temperature plus 80° C. The drying is generally conducted under normal pressure or reduced pressure and in a stationary state or in blast.

The electrophotographic photoconductor according to the present invention can be used in known electrophotographic processes and can be suitably used in common electrophotographic processes including a charging, exposure, development, transfer, or fixing process. Namely, the electrophotographic photoconductor according to the present invention can be used in copiers, printers, and fax machines having these electrophotographic processes.

Here, as the charging process, a positive charging process that positively charges the photoconductor to a positive pole and a negative charging process that negatively charges the photoconductor to a negative pole are known. The photoconductor according to the present invention can be used in the negative charging process, but since it shows a particularly high sensitivity in the positive charging process, the photoconductor is preferably used in the positive charging process. In particular, when the electrophotographic photoconductor is a single-layer type including a photosensitive layer containing a charge-generating material, a charge-transporting material, and a resin binder, wherein the charge-transporting material contains an electron-transporting material and a hole-transporting material and the electron-transporting material contains at least a compound represented by the Formula (I) according to the present invention, the electrophotographic photoconductor can exhibit a high sensitivity in the positive charging process.

As chargers for the charging process, a noncontact charger using corotron or scorotron and a charger of a roller or brush-shape conducting the charging in contact (or contiguity) with the photoconductor are known. The photoconductor according to the present invention can be used in both processes utilizing either of the chargers.

Light sources used in the exposure process usually have a wavelength band in which the photoconductor has sensitivity. White light sources such as a halogen lamp and a fluorescent lamp, laser light sources, and light-emitting diode (LED) light sources are preferable. In particular, a semiconductor laser light or LED light of about 600 to 800 nm is more preferable when phthalocyanine is used as the charge-generating material. Additionally, a semiconductor laser light or LED light of 450 nm or less can be used when a compound having absorption at 450 nm or less is used as the charge-generating material. Furthermore, an internal exposure system can be applied by using a permeable material as the electrically conductive substrate of the photoconductor.

As principal development processes, a dry development system using dry toner and a liquid (wet) development system using liquid toner are known. The photoconductor according to the present invention can be used in both systems. In the liquid development system, a known method for preventing the components of the photoconductor from dissolving into the solvent contained in the liquid toner is desirably used.

Additionally, as the development processes, a reversal development system that develops toner in exposure areas and a normal development system that develops toner in non-exposure areas are known. In particular, the reversal development system is preferably used when phthalocyanine is used as the charge-generating material.

Some known electrophotographic processes have, for the purpose of removing or scattering remaining non-transferred toner in the photoconductor, a cleaning process after the transfer process, but some electrophotographic processes do not have the cleaning process. The photoconductor according to the present invention can be used in both processes.

Additionally, some known electrophotographic processes have, for the purpose of removing remaining charges or equating the surface potential, an electricity-removing process by exposure after the transfer process, but some electrophotographic processes do not have the electricity-removing process. The photoconductor according to the present invention can be used in both processes.

The electrophotographic apparatus according to the present invention includes the electrophotographic photoconductor of the present invention and performs a charging process by the positive charging process, but electrophotographic processes other than the charging process can be performed without any limitation.

### EXAMPLES

Examples of the present invention will now be described.

Apparatuses used for measurements in the following Synthesis Examples are as follows:

Melting point measurement: Micro-melting point apparatus (Yanagimoto Manufacturing Co., Ltd.), Measurement values are not compensated.

200 MHz ¹H-NMR spectral analysis: GEMINI-2000 (Varian Inc.)

500 MHz ¹H-NMR spectral analysis: DRX-500 (Bruker Co., Ltd.)

MS spectral analysis; POLARIS-Q (Thermo Finnigan)

### Synthesis Example 1

### Synthesis of a Compound Shown by the Structural Formula (I-1)

(1) Synthesis of 4,4′-Oxybis(Hydrazinobenzene) Hydrochloride (The Formula (IV), A=O, R¹¹═R¹²═H, and n=m=1)

Water (100 mL) and 35 to 37% hydrochloric acid (150 mL) were put into a 500-mL four-neck flask, and then oxydianiline (20 g (0.10 mol), the formula (II), A=O, R¹¹═R¹²═H, and n=m=1: Tokyo Kasei Kogyo Co., Ltd.) was added. Additionally, a solution prepared by dissolving sodium nitrite (15.2 g (0.22 mol)) in water (50 mL) was gradually put into the flask at −10 to 0° C., and the resulting mixture was stirred at a temperature not exceeding 0° C. for 1 hr to prepare a bisdiazonium salt solution. 35 to 37% hydrochloric acid (300 mL) was put into a 1-L four-neck flask, and tin (II) chloride dehydrate (148.6 g (0.66 mol)) was added and dissolved. Then, the above-prepared bisdiazonium salt solution was dropwise added into the resulting solution for 30 min at a temperature of −10 to −5° C. Then, the temperature of the resulting mixture was increased from −5° C. to a room temperature under stirring overnight. The precipitated product was separated by filtration and was recrystallized from 1% hydrochloric acid (400 mL) to yield 15.8 g 4,4′-oxybis(hydrazinobenzene) hydrochloride.

Yield: 52.2%, mp 181 to 184° C.

¹H-NMR (500 MHz, DMSO-D₆); δ 6.92 (d, J=8.9 Hz, 4H), 7.05 (d, J=8.9 Hz, 4H), 10.15 (brs, 6H).

MS (Direct-EI); 230, 214, 200, 184, 169.

(2) Synthesis of a Compound Shown by Structural Formula (I-1)

4,4′-Oxybis(hydradinobenzene)hydrochloride (7.0 g (23.1 mmol), the formula (IV), A=O, R¹¹═R¹²═H, and n=m=1), 3,5-di-tert-butyl-4-hydroxybenzaldehyde (10.8 g (46.1 mmol), the formula (V), R¹═R²=tert-butyl, R⁵═R⁶═R═H, Tokyo Kasei Kogyo Co., Ltd.), and sodium acetate (4.3 g (52.4 mmol)) were put into a 200-mL four-neck flask and reacted in ethanol (100 mL) under nitrogen at room temperature overnight. About a half of the ethanol was collected under reduced pressure, and water (100 mL) was added to the flask and the resulting mixture was stirred at a room temperature for 30 min. The product was separated by filtration and was dried to yield 15.0 g crude bishydrazone (the formula (VI), A=O, R¹═R²═R³═R⁴=tert-butyl. R⁵═R⁶═R⁷═R⁸═R⁹═R¹⁰═R¹¹═R¹²═H, and n=m=1).

This 15.0 g of crude bishydrazone was dissolved in toluene (150 mL), and 2,3-dichloro-5,6-dicyano-1,4-benzoquinone (9.5 g (41.9 mmol)) was gradually added to the resulting solution and reacted at a room temperature for 2 hr. The precipitate was removed by filtration and the filtrate was concentrated. The concentrated filtrate was purified by silica gel column chromatography using toluene as an eluting solvent to yield 7.0 g of the compound represented by the structural formula (I-1), and further recrystallized from hexane to yield 5.3 g of the compound (I-1).

Yield: 38.9%, mp 196 to 199° C.

¹H-NMR (500 MHz, CDCl₃); δ 1.35 (s, 18H), 1.40 (s, 18H), 7.12 (s, J=2.2 Hz, 2H), 7.21 (d, J=8.8 Hz, 4H), 7.67 (s, 2H), 7.97 (d, J=8.8 Hz, 4H), 8.30 (d, J=2.2 Hz, 2H).

MS (Direct-EI); 658, 602.

### Synthesis Example 2

### Synthesis of a Compound Shown by the Structural Formula (I-3)

(1) Synthesis of 4,4′-Oxybis(2,6-Bromoaniline) (The Formula (II), A=O, R¹¹′R¹²═Br, and n=m=2)

In a 200-mL four-neck flask, 4,4′-oxydianiline (5.0 g (25.0 mmol), the formula (II), A=O, R¹¹═R¹²═H, and n=m=1, Tokyo Kasei Kogyo-Co., Ltd.) was dissolved in acetic acid (35 mL), and 1,4-dioxane (35.2 g (399.5 mmol)) was added. Under ice cooling, bromine (18.4 g (72.4 mmol)) was dropped into the flask for 1 hr. Then, after the addition of water, extraction with toluene was conducted. The organic phase was washed with water, a sodium hydroxide solution, and then water, and then concentrated. The concentrated organic phase was purified by silica gel column chromatography using toluene as an eluting solvent to yield 11.7 g 4,4′-oxybis(2,6-dibromoaniline).

Yield: 90.8%, mp 166 to 167° C.

¹H-NMR (200 MHz, CDCl₃); δ 4.39 (brs, 4H), 7.07 (s, 4H).

MS (Direct-EI); 515, 409, 355, 266.

(2) Synthesis of Bishydrazone (the Formula (VI), A=O, R¹═R²═R³═R⁴=tert-butyl, R⁵═R⁶═R⁷═R⁸═R⁹═R¹⁰═H, R¹¹═R¹²=Br, and n=m=2)

4,4′-Oxybis(2,6-dibromoaniline) (11.0 g (21.3 mmol), the formula (II), A=O, R¹¹═R¹²=Br, and n=m=2) and 35 to 37% hydrochloric acid (110 mL) were put into a 500-mL four-neck flask. The mixture was stirred at a room temperature for 1 hr until crystalline amine was slurried. After cooling to −10° C., a solution prepared by dissolving sodium nitrite (3.1 g (44.8 mmol)) in water (9.3 mL) was gradually added and stirred at a temperature not exceeding OC for 1 hr to prepare a bisdiazonium salt solution. Tin (II) chloride dihydrate (21.2 g (93.8 mmol)), tin (506.3 mg (4.27 mmol)), and 35 to 37% hydrochloric acid (44 mL) were put into a 500-mL four-neck flask and stirred at 40° C. for 1 hr to dissolve the tin. After cooling to −10 to −5° C., the above-prepared bisdiazonium salt solution was dropwise added for 30 min. Then, the mixture was stirred for 1 hr. The precipitated product was separated by filtration and was recrystallized from a 1% hydrochloric acid solution (250 mL) to yield 9.16 g bishydrazine hydrochloride (the formula (IV), A=O, R¹¹═R¹²═Br, and n=m=2).

The above-prepared bishydrazine hydrochloride and N,N′-dimethyl formamide (138 mL) were put into a 500-mL four-neck flask under nitrogen atmosphere and 3,5-di-tert-butyl-4-hydroxybenzaldehyde (28.0 g (119.4 mmol), the formula (V), R¹═R²=tert-butyl, R⁵═R⁶═R⁹═H, Tokyo Kasei Kogyo Co., Ltd.) and sodium acetate (8.7 g (140.7 mmol)) were added and stirred at a room temperature for 1 hr. After the addition of toluene, the organic phase was washed with water and concentrated. The concentrated organic phase was purified by silica gel column chromatography using chloroform as an eluting solvent to yield 6.85 g bishydrazone.

Yield: 32.8% (an oily substance)

¹H-NMR (200 MHz, CDCl₃); δ 1.46 (s, 36H), 5.36 (s, 2H), 7.26 (s, 4H), 7.35 (s, 2H), 7.50 (s, 4H), 7.73 (s, 2H).

MS (Direct-EI); 979.

(3) Synthesis of a Compound Shown by the Structural Formula (I-3)

Bishydrazone (10.1 g (10.3 mmol), the formula (VI), R¹═R²═R³═R⁴ tert-butyl, R⁵═R⁶═R⁷═R⁸═R⁹═R¹⁰═H, A=O, R¹¹═R¹²=Br, and n=m=2), toluene (200 mL), and chloroform (100 mL) were put into a 500-mL four-neck flask and dissolved. To the solution, manganese dioxide (3.59 g (41.3 mmol)) was added. The mixture was stirred at a room temperature overnight. The precipitate was removed by filtration and the filtrate was concentrated. The concentrated filtrate was purified by silica gel column chromatography using toluene as an eluting solvent, and was further recrystallized from a toluene-hexane solvent mixture to yield 8.23 g of the compound represented by the structural formula (I-3).

Yield: 81.8%, mp 228 to 231° C.

¹H-NMR (500 MHz, CDCl₃); δ 1.35 (s, 36H), 7.18 (d, J=2.2 Hz, 2H), 7.41 (s, 4H), 7.71 (s, 2H), 8.32 (d, J=2.2 Hz, 2H).

MS (Direct-EI); 974, 918.

### Synthesis Example 3

### Synthesis of a Compound Shown by the Structural Formula (I-41)

(1) Synthesis of 4-Hydrazinophenyl Sulfonate (The Formula (IV), A=SO₂, R¹¹═R¹²═H, and n=m=1)

Into a 500-mL four-neck flask, 35 to 37% hydrochloric acid (300 mL) was put and 4-aminophenyl sulfone (20 g (0.081 mol), the formula (II), A=SO₂, R¹¹═R¹²═H, and n=m=1, Tokyo Kasei Kogyo Co., Ltd.) was added. To the mixture, a solution prepared by dissolving sodium nitrite (11.5 g (0.167 mol)) in water (40 mL) was gradually added at −10 to 0° C. and stirred at a temperature not exceeding 0° C. for 1 hr to prepare a bisdiazonium salt solution. Into a 1-L four-neck flask, 35 to 37% hydrochloric acid (200 mL) was put and tin (II) chloride dihydrate (70 g (0.310 mol)) was added and dissolved. Then, the above-prepared bisdiazonium salt solution was dropwise added into the resulting solution for 30 min at a temperature of −10 to −5° C. Then, the mixture was stirred at −5° C. The precipitated product was separated by filtration and was recrystallized from water (90 mL.) to yield 11.2 g 4-hydrazinophenyl sulfone.

Yield: 39.4%, mp 190 to 191° C.

¹H-NMR (500 MHz, DMSO-D₆); δ 7.05 (d, J=7.1 Hz, 4H), 7.77 (d, J=7.1 Hz, 4H), 8.90 (brs, 2H), 10.25 (brs, 4H).

MS (Direct-EI); 278, 261, 232, 108.

(2) Synthesis of a Compound Shown by the Structural Formula (I-41)

4-Hydrazinophenyl sulfone (5.0 g (14.2 mmol), the ormula (IV), A=SO₂, R¹¹′R¹²═H, and n=m=1), 3,5-di-tert-butyl-4-hydroxybenzaldehyde (6.7 g (28.6 mmol), the formula (V), R¹═R²=tert-butyl, R⁵═R⁶═R⁹═H, Tokyo Kasei Kogyo Co., Ltd.), and pyridine (3 mL (37.1 mmol)) were put into a 200-mL four-neck flask, and the mixture was refluxed in isopropanol (60 mL) under nitrogen for 3 hr. The mixture was poured into water and was extracted with toluene/diethyl ether. The organic phase was dried over magnesium sulfate and concentrated to yield 11.3 g crude bishydrazone (the formula (VI), A=SO₂, R¹═R²═R³═R⁴=tert-butyl, R⁵═R⁶═R⁷═R⁵═R⁹═R¹⁰═R¹¹═R¹²═H, and n=m=1).

The crude bishydrazone (11.3 g) was dissolved in methylene chloride (120 mL). To the solution, manganese dioxide (5 g (57.5 mmol)) was gradually added. The mixture was reacted at a room temperature overnight and further reacted under reflux for 30 min. The precipitate was removed by filtration and the filtrate was concentrated. The concentrated filtrate was purified by silica gel column chromatography using toluene/ethyl acetate (8:1) as an eluting solvent to yield 7.2 g of the compound represented by the structural formula (I-41), and further recrystallized from toluene to yield 6.42 g of the compound (I-41).

Yield: 63.9%, mp 258 to 261° C.

¹H-NMR (500 MHz, CDCl₃); δ 1.34 (s, 18H), 1.36 (s, 18H), 7.10 (s, 2H), 7.68 (s, 2H), 7.97 (d, J=7.0 Hz, 4H), 8.12 (d, J=7.0 Hz, 4H), 8.27 (s, 2H).

MS (Direct-EI); 706, 650, 231, 216, 188, 172.

### Photoconductor Example 1

A plate-shaped photoconductor for evaluation of electric property and a drum-shaped photoconductor for evaluation of printing were produced. Hereinafter, “part(s)” indicates part(s) by weight.

An undercoat-layer solution prepared as described below was applied on the external surfaces of an aluminum plate (3 cm×10 cm, thickness: 1 mm) and an aluminum cylinder (external diameter: 30 mm, length: 247.5 mm, thickness: 0.75 mm) by dip coating. The solvent was removed by drying the plate and the cylinder at 100° C. for 60 min to form undercoat layers having a thickness of 0.3 μm.

(Preparation of Undercoat-Layer Solution)

(a1) Soluble nylon (Amilan CM-8000: Toray Industries, Inc.) 3 parts (30 g)

The material (a1) for the undercoat layer was agitated with in 97 parts of a methanol/methylene chloride (in a volume ratio of 5:5) solvent mixture (970 g) for dissolving in it to prepare the undercoat-layer solution.

Then, a dispersion liquid for the single-layer photosensitive layer, which was prepared as described below, was applied on the undercoat layers of the plate by dip coating and the cylinder by ring coating. The solvent was removed by drying the plate and the cylinder at 10° C. for 60 min to form single-layer photosensitive layers having a thickness of 30 μm. Thus, electrophotographic photoconductors were produced.

(Preparation of Dispersion Liquid for Single-Layer Photosensitive Layer)

(b1) Charge-generating material: x-type metal-free phthalocyanine (see FIG. 2 of Japanese Unexamined Patent Application Publication No. 2001-228637) 0.2 parts (0.1 g)

(b2) Hole-transporting material: a styryl compound represented by the following formula ((HT1-101) in Japanese Unexamined Patent Application Publication No. 2001-314969) 8 parts (4 g) 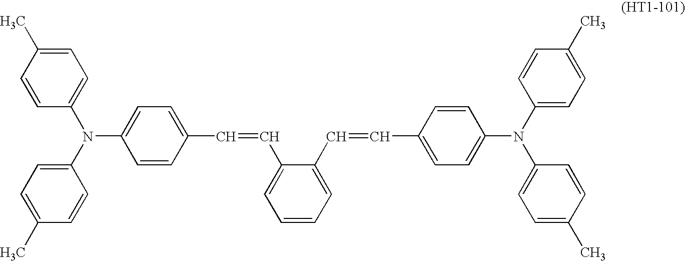
(b3) Electron-transporting material: a compound shown by the formula (I-1) [Synthesis Example 1] 5 parts (2.5 g)
(b4) Antioxidant: 3,5-di-tert-butyl-4-hydroxytoluene (BHT) 1 part (0.5 g)
(b5) Silicone oil (KF-50: Shin-Etsu Chemical Co., Ltd.) 0.01 parts (0.005 g)
(b6) Resin binder: Bisphenol z-type polycarbonate resin (Panlite TS2050: Teijin Chemicals Ltd.) ((BD1-1) disclosed in Japanese Unexamined Patent Application Publication No. 2000-314969) 7 parts (3.5 g)

The materials (b1) to (b6) for a photosensitive layer, 100 parts of a methylene chloride solvent (50 g), and 50 g of stainless steel beads having a diameter of 3 mm were put into a 100-mL plastic bottle. The mixture was dispersed by using Paint Conditioner Model 5400 (Red Devil Equipment Co.: USA) for 60 min, and then the stainless steel beads were removed. Thus, the dispersion liquid for the single-layer photosensitive layer was prepared.

### Photoconductor Example 2

A photoconductor was produced as in Photoconductor Example 1 except that 5 parts of a compound shown by the formula (I-3) in the Synthesis Example 2 was used instead of 5 parts of the compound shown by the formula (I-1) as the electron-transporting material in the components of the dispersion liquid for the single-layer photosensitive layer used in Photoconductor Example 1.

### Photoconductor Example 3

A photoconductor was produced as in Photoconductor Example 1 except that 5 parts of a compound shown by the formula (I-41) in the Synthesis Example 3 was used instead of 5 parts of the compound shown by the formula (I-1) as the electron-transporting material in the components of the dispersion liquid for the single-layer photosensitive layer used in Photoconductor Example 1.

### Photoconductor Example 4

A photoconductor was produced as in Photoconductor Example 2 except that 7 parts instead of 8 parts of styryl compound shown by the formula (HT1-101), 2 parts instead of 5 parts of the compound shown by the formula (I-3) as the electron-transporting material, and 10 parts instead of 7 parts of bisphenol z-type polycarbonate resin were used as the components of the dispersion liquid for the single-layer photosensitive layer used in Photoconductor Example 2.

### Photoconductor Example 5

A photoconductor was produced as in Photoconductor Example 1 except that 8 parts of a styryl compound shown by the following formula (HT2-2) of Japanese Unexamined Patent Application Publication No. 2000-314969 instead of 8 parts of the styryl compound shown by the formula (HT1-101) as the hole-transporting material in the components of the dispersion liquid for the single-layer photosensitive layer used in Photoconductor Example 1.

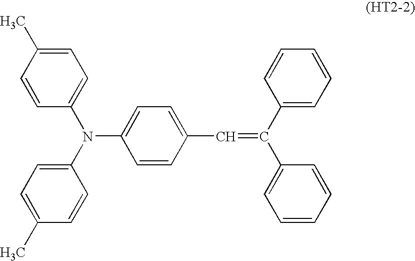

### Photoconductor Example 6

A photoconductor was produced as in Photoconductor Example 1 except that 8 parts of a diamine compound shown by the following formula (HT-11) of Japanese Unexamined Patent Application Publication No. 2000-314969 instead of 8 parts of the styryl compound shown by the formula (HT1-101) as the hole-transporting material in the components of the dispersion liquid for the single-layer photosensitive layer used in Photoconductor Example 1.

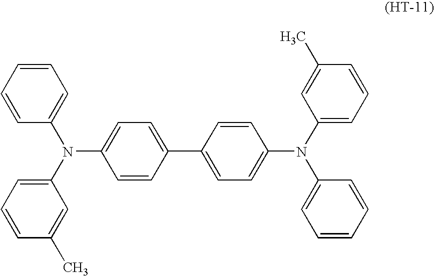

### Photoconductor Example 7

A photoconductor was produced as in Photoconductor Example 1 except that 0.3 parts instead of 0.2 parts of x-type metal-free phthalocyanine was used as the charge-generating material in the components of the dispersion liquid for the single-layer photosensitive layer used in Photoconductor Example 1.

### Photoconductor Example 8

A photoconductor was produced as in Photoconductor Example 1 except that 0.3 parts of α-type titanyl phthalocyanine (see FIG. 3 of Japanese Unexamined Patent Application Publication No. 2001-228637) was used instead of 0.2 parts of x-type metal-free phthalocyanine as the charge-generating material in the components of the dispersion liquid for the single-layer photosensitive layer used in Photoconductor Example 1.

### Photoconductor Example 9

A photoconductor was produced as in Photoconductor Example 1 except that 0.1 parts of y-type titanyl phthalocyanine (see FIG. 4 of Japanese Unexamined Patent Application Publication No. 2001-228637) was used instead of 0.2 parts of x-type metal-free phthalocyanine as the charge-generating material in the components of the dispersion liquid for the single-layer photosensitive layer used in Photoconductor Example 1.

### Photoconductor Example 10

A photoconductor was produced as in Photoconductor Example 1 except that 0.1 parts of amorphous titanyl phthalocyanine (see FIG. 5 of Japanese Unexamined Patent Application Publication No. 2001-228637) was used instead of 0.2 parts of x-type metal-free phthalocyanine as the charge-generating material in the components of the dispersion liquid for the single-layer photosensitive layer used in Photoconductor Example 1.

### Photoconductor Example 11

A photoconductor was produced as in Photoconductor Example 1 except that 0.2 parts of a bisazo compound shown by the following formula (CG1-1) was used instead of 0.2 parts of x-type metal-free phthalocyanine as the charge-generating material in the components of the dispersion liquid for the single-layer photosensitive layer used in Photoconductor Example 1.

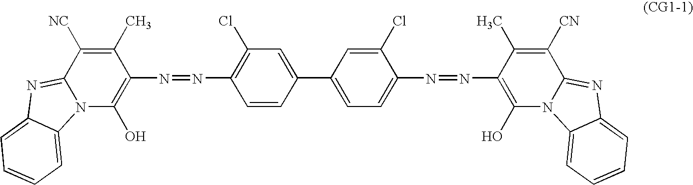

### Photoconductor Example 12

A photoconductor was produced as in Photoconductor Example 1 except that 0.2 parts of a bisazo compound shown by the above-mentioned formula (CG1-1) was further added as an electron-transporting material to the components of the dispersion liquid for the single-layer photosensitive layer used in Photoconductor Example 1.

### Photoconductor Comparative Example 1

A photoconductor was produced as in Photoconductor Example 1 except that 5 parts of a stilbenequinone compound (Tokyo Kasei Kogyo Co., Ltd.) shown by the following formula (ET-1) was used instead of 5 parts of the compound shown by the formula (I-1) as the electron-transporting material in the components of the dispersion liquid for the single-layer photosensitive layer used in Photoconductor Example 1.

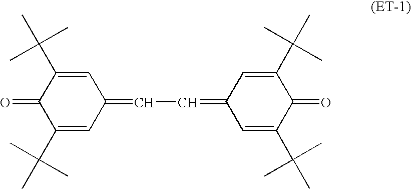

### Photoconductor Comparative Example 2

A photoconductor was produced as in Photoconductor Example 1 except that 5 parts of a diphenoquinone compound shown by the following formula (ET-2) was used instead of 5 parts of the compound shown by the formula (I-1) as the electron-transporting material in the components of the dispersion liquid for the single-layer photosensitive layer used in Photoconductor Example 1.

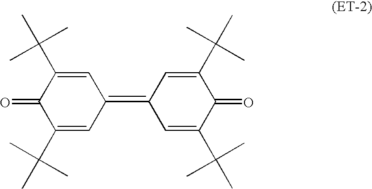

### Photoconductor Comparative Example 3

A photoconductor was produced as in Photoconductor Example 1 except that 5 parts of a compound shown by the following formula (ET-3) was used instead of 5 parts of the compound shown by the formula (I-1) as the electron-transporting material in the components of the dispersion liquid for the single-layer photosensitive layer used in Photoconductor Example 1.

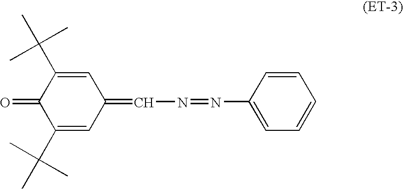

### Photoconductor Comparative Example 4

A photoconductor was produced as in Photoconductor Example 1 except that 5 parts of a compound shown by the following formula (ET-4) was used instead of 5 parts of the compound shown by the formula (I-1) as the electron-transporting material in the components of the dispersion liquid for the single-layer photosensitive layer used in Photoconductor Example 1. 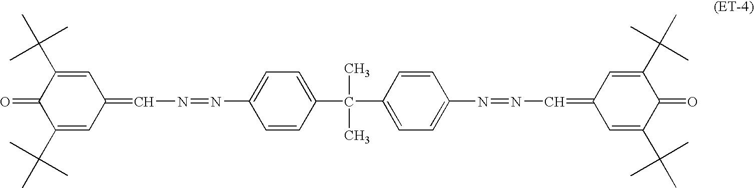
Evaluation of Photoconductor Examples 1 to 12 and Photoconductor Comparative Examples 1 to 4

Electric properties of the plate-shaped photoconductors were evaluated using an electrostatic copying paper testing apparatus EPA-8100 manufactured by Kawaguchi Electric Works Co., Ltd.

The photoconductors were charged in the dark under the conditions of a temperature of 24° C. and a humidity of 50% so that the surface potential was about +700 V, and surface potential retentions after 5 sec, Vk₅, were calculated using the following equation:
Retention ratio Vk₅(%)=(V₅/V₀)×100

(wherein V₀ denotes a surface potential right after the charging, and V₅ denotes a surface potential after 5 seq).

Then, the surface potential was adjusted to +600 V and the photoconductors were exposed for 5 see to monochromatic 780 nm (except 550 nm in the Photoconductor Example 11) filtered light with an intensity of 1.0 μW/cm² from a halogen lamp. An exposure amount necessary for reducing the surface potential to a half (+300 V) was determined as a sensitivity, E_{1/2} (μJ/cm²), and the surface potential at 5 sec after the exposure was determined as a residual potential, Vr (V).

Appearances of the drum-shaped photoconductors were visually observed.

Table 1 shows the results of these evaluations.

TABLE 1

| | Retention | Sensitivity | Residual | |
|---|---|---|---|---|
| | ratio Vk₅ | E_{1/2} | potential | Photoconductor |
| | (%) | (μJ/cm²) | Vr (V) | appearance |
| Photoconductor | 86.5 | 0.31 | 40 | Good |
| Example 1 | | | | |
| Photoconductor | 89.1 | 0.35 | 45 | Good |
| Example 2 | | | | |
| Photoconductor | 80.3 | 0.39 | 50 | Good |
| Example 3 | | | | |
| Photoconductor | 85.9 | 0.43 | 57 | Good |
| Example 4 | | | | |
| Photoconductor | 88.3 | 0.36 | 43 | Good |
| Example 5 | | | | |
| Photoconductor | 78.4 | 0.46 | 58 | Good |
| Example 6 | | | | |
| Photoconductor | 78.9 | 0.29 | 37 | Good |
| Example 7 | | | | |
| Photoconductor | 81.0 | 0.27 | 36 | Good |
| Example 8 | | | | |
| Photoconductor | 80.4 | 0.26 | 39 | Good |
| Example 9 | | | | |
| Photoconductor | 83.2 | 0.33 | 45 | Good |
| Example 10 | | | | |
| Photoconductor | 84.2 | 0.46 | 59 | Good |
| Example 11* | | | | |
| Photoconductor | 79.0 | 0.25 | 37 | Good |
| Example 12 | | | | |
| Photoconductor | 71.3 | 0.57 | 89 | Precipitation |
| Comparative | | | | |
| Example 1 | | | | |
| Photoconductor | 69.7 | 0.65 | 98 | Precipitation |
| Comparative | | | | |
| Example 2 | | | | |
| Photoconductor | 84.2 | 0.38 | 55 | Good |
| Comparative | | | | |
| Example 3 | | | | |
| Photoconductor | 78.3 | 0.47 | 66 | Good |
| Comparative | | | | |
| Example 4 | | | | |

*Exposure light: 550 nm

As shown in Table 1, the photoconductors of the Photoconductor Comparative Examples 3 and 4 have a relatively good retention ratio, sensitivity, and residual potential, but they are slightly inferior to the photoconductor of the Photoconductor Example 1 which was produced by the same manner except the electron-transporting material.

For the evaluation of durability against actual printing the drum-shaped photoconductors were mounted on Laser printer HL-1850 manufactured by Brother Industries, Ltd., and a black solid image, a white solid image, and a halftone image were printed under the conditions of a temperature of 22° C. and a humidity of 44%. Then, 5000 pages of images having a printing ratio of about 5% were printed. Then, a black solid image, a white solid image, and a halftone mage were printed again in order to evaluate the images after the printing of the 5000 pages.

As a result, in the photoconductors of the Photoconductor Examples 1 to 6 and 10 and the Photoconductor Comparative Examples 3 and 4, good images were observed in both the initial and after the printing of the 5000 pages. On the other hand, in the photoconductors of the Photoconductor Comparative Examples 1 and 2, image unevenness, which is thought to be caused by precipitation, was observed in the initial halftone images. The photoconductor of the Photoconductor Example 11 did not have a sufficient sensitivity to the laser wavelength band (near 780 nm) of the laser printer and was inadequate to this laser printer. Since the sensitivities of the photoconductors of the Photoconductor Examples 7, 8, 9, and 12 were too high, these photoconductors were slightly inadequate to this laser printer and their halftone images tended to be crashed.

What is claimed is:

1. A quinone compound comprising a structure represented by Formula (I): 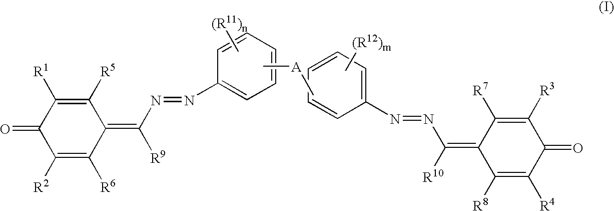
wherein R₁, R², R³, R⁴, R⁵, R⁶, R⁷ and R⁸ may be the same or different and each denotes hydrogen or a substituted or unsubstituted alkyl group having 1 to 6 carbon atoms; R⁹ and R¹⁰ may be the same or different and each denotes hydrogen, a substituted or unsubstituted alkyl group having 1 to 6 carbon atoms, a substituted or unsubstituted aryl group, or a substituted or unsubstituted heterocyclic group; R¹ and R⁵, R² and R⁶, R³ and R⁷, and R⁴ and R⁸ may bind to each other to form a ring, respectively; R¹¹ and R¹² may be the same or different and each denotes a halogen, a substituted or unsubstituted alkyl group having 1 to 6 carbon atoms, an alkoxy group having 1 to 6 carbon atoms, an alkyl halide group having 1 to 6 carbon atoms, a nitro group, a substituted or unsubstituted aryl group, or a substituted or unsubstituted heterocyclic group; n and m each denotes an integer of 0 to 4; when n Is larger than 1, the plurality of R¹¹ may bind to each other to form a ring; when m is larger than 1, the plurality of R¹² may bind to each other to form a ring; A denotes oxygen or SO₂; and the substituent is a halogen, an alkyl group having 1 to 6 carbon atoms, an alkoxy group having 1 to 6 carbon atoms, an alkyl halide group having 1 to 6 carbon atoms, a nitro group, an aryl group, or a heterocyclic group.

2. An electrophotographic photoconductor comprising a photosensitive layer containing a charge-generating material and a charge-transporting material on an electrically conductive substrate, wherein the photosensitive layer contains at least a compound represented by Formula (I): 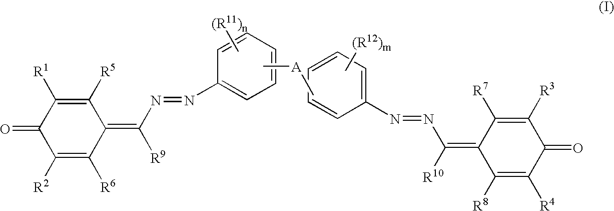
wherein R¹, R², R³, R⁴, R⁵, R⁶, R⁷ and R⁸ may be the same or different and each denotes hydrogen or a substituted or unsubstituted alkyl group having 1 to 6 carbon atoms; R⁹ and R¹⁰ may be the same or different and each denotes hydrogen, a substituted or unsubstituted alkyl group having 1 to 6 carbon atoms, a substituted or unsubstituted aryl group, or a substituted or unsubstituted heterocyclic group; R^{1 and R}⁵, R² and R⁶, R³ and R⁷, and R⁴ and R⁸ may bind to each other to form a ring, respectively; R¹¹ and R¹² may be the same or different and each denotes a halogen, a substituted or unsubstituted alkyl group having 1 to 6 carbon atoms, an alkoxy group having 1 to 6 carbon atoms, an alkyl halide group having 1 to 6 carbon atoms, a nitro group, a substituted or unsubstituted aryl group, or a substituted or unsubstituted heterocyclic group; n and m each denotes an integer of 0 to 4; when n is larger than 1, the plurality of R¹¹ may bind to each other to form a ring; when m is larger than 1, the plurality of R¹² may bind to each other to form a ring; A denotes oxygen or SO₂; and the substituent is a halogen, an alkyl group having 1 to 6 carbon atoms, an alkoxy group having 1 to 6 carbon atoms, an alkyl halide group having 1 to 6 carbon atoms, a nitro group, an aryl group, or a heterocyclic group.

3. The electrophotographic photoconductor according to claim 2, wherein the photosensitive layer is a single-layer photosensitive layer containing a charge-generating material, a charge-transporting material, and a resin binder, wherein the charge-transporting material contains an electron-transporting material and a hole-transporting material, wherein the electron-transporting material contains at least a compound comprising a structure represented by the Formula (I).

4. The electrophotographic photoconductor according to claim 2, wherein the photosensitive layer contains a hole-transporting material, wherein the hole-transporting material contains a styryl compound.

5. The electrophotographic photoconductor according to claim 2, wherein the photosensitive layer contains a charge-generating material, wherein the charge-generating material contains a phthalocyanine compound.

6. An electrophotographic apparatus, which comprises the electrophotographic photoconductor according to claim 2 and a positive charging system.
